(19) United States
(12) Patent Application Publication
(10) Pub. No.: US 2023/0270148 A1
(43) Pub. Date: Aug. 31, 2023

(54) BITTER BLOCKERS AND RELATED METHODS OF USE

(71) Applicants: Conagen Inc., Bedford, MA (US); Sweegen, Inc., Rancho Santa Margarita, CA (US)

(72) Inventors: Shari Mahon, Florence, KY (US); Rui Zhou, Acton, MA (US); Steven Chen, Coto de Caza, CA (US)

(73) Assignees: Conagen Inc., Bedford, MA (US); Sweegen, Inc., Rancho Santa Margarita, CA (US)

(21) Appl. No.: 18/046,698

(22) Filed: Oct. 14, 2022

Related U.S. Application Data
(63) Continuation of application No. PCT/US2021/027792, filed on Apr. 16, 2021.
(60) Provisional application No. 63/172,284, filed on Apr. 8, 2021.
(60) Provisional application No. 63/172,294, filed on Apr. 8, 2021.
(60) Provisional application No. 63/011,312, filed on Apr. 17, 2020.

(51) Int. Cl. A23L 27/00; A23L 2/56; A23L 33/105; A23F 3/40; A61K 9/00; A61K 47/26; C12P 17/06

(52) U.S. Cl. CPC A23L 27/86; A23L 2/56; A23L 33/105; A23F 3/405; A61K 9/0095; A61K 47/26; C12P 17/06

(57) ABSTRACT

The present invention relates, at least in part, to compounds and compositions that can be used to mask, block, or reduce the bitter taste present in various orally consumable products. The present invention also relates to methods of using bitterness blocking compounds and compositions to mask the bitterness of various orally consumable products, hence making such orally consumable products more palatable. The present invention further relates to an orally consumable product with reduced bitterness.

### RELATED APPLICATIONS

This application claims the benefit under 35 U.S.C. § 119(e) to U.S. Provisional Application No. 63/011,312, filed Apr. 17, 2020, and entitled “BITTER BLOCKERS AND RELATED METHODS OF USE,” U.S. Provisional Application No. 63/172,284, filed Apr. 8, 2021, and entitled “BITTER BLOCKERS AND RELATED METHODS OF USE,” and U.S. Provisional Application No. 63/172,294, filed Apr. 8, 2021, and entitled “BITTER BLOCKERS AND RELATED METHODS OF USE,” the entire contents of each of which are incorporated herein by reference.

### FIELD OF THE INVENTION

The field of the invention relates to compounds that can be used to mask, block, or reduce the bitter taste present in various consumable products containing a bitter-tasting substance.

### BACKGROUND OF THE INVENTION

Many drugs and certain foods taste bitter or otherwise impart a bitter off-taste or aftertaste. Various strategies have been developed to mask bitterness to encourage treatment compliance and consumption of such bitter-tasting drugs and foods.

During the experience of “tasting,” several physiological and psychological events occur simultaneously. Anatomically, taste cells reside within specialized structures called taste buds, which are located on the tongue and soft palate. The majority of taste buds are located within papillae, which are the tiny projections on the surface of the tongue that give it its velvety appearance. Taste buds are onion-shaped structures of between 50 and 100 taste cells, each of which possesses finger-like projections called microvilli that protrude through an opening at the top of the taste bud called the taste pore. Chemicals from food known as tastants dissolve in saliva and contact the taste cells via the taste pore. There, they either interact with surface proteins of the cells called taste receptors (for sweet and bitter tastes), or they interact with pore-like proteins called ion channels (for salty and sour tastes). These interactions cause electrical changes within the taste cells that trigger them to send chemical signals that translate into neurotransmission to the brain. The electrical responses that send the signal to the brain are a result of a varying concentration of charged atoms or ions within the taste cell. These cells normally have a net negative charge. Tastants alter this state by using varying means to increase the concentration of positive ions within the taste cell. This depolarization causes the taste cells to release neurotransmitters, prompting neurons connected to the taste cells to relay electrical messages to the brain.

In the case of a bitter taste, stimuli act by binding to G-protein coupled receptors on the surface of the taste cell. This then prompts the protein subunits of alpha, beta, and gamma to split and activate a nearby enzyme. This enzyme then converts a precursor within the cell into a “second messenger”. The second messenger causes the release of calcium ions (Ca²⁺) from the endoplasmic reticulum of the taste cell. The resulting build-up of calcium ions within the cell leads to depolarization and neurotransmitter release. The signal now sent to the brain is interpreted as a bitter taste.

Generally speaking, one class of stimuli will be most effective in eliciting the highest frequency discharge because receptor specificity is considered relative as opposed to an all-or-none response. In other words, the differences between stimuli are not so much a difference between firing and non-firing of the neurons, but is in fact the differences in the amount of firing of the neurons. This consideration would explain, for example, why a sweet compound might reduce the perception of a hitter compound. The overall taste perception of the brain is dependent upon the amount of firing of the receptors. For example, by causing the receptors of sweetness to become engaged while the bitterness receptors are engaged can reduce the net effect of both taste sensations to the brain. Accordingly, a method of diminishing the overall response to one stimulus would be to introduce additional stimuli, such that central cognitive interactions lead to one strong taste or aroma reducing the perception of the other in the brain. Without wishing to be bound by any particular theory, compounds and compositions that can be used to mask, block, or reduce the bitterness of a hitter-tasting substance can do so by (i) physically coating taste receptors within the taste buds, thereby impeding or blocking direct contact between the taste receptors and the bitter-tasting substance, (ii) competing with the bitter-tasting substance at the ion channels in the taste buds, and/or (iii) competing with the bitter-tasting substance for the remaining, available taste receptors within the taste buds.

Various compounds have been used by the food and drug industry as bitter blockers. However, most bitter blockers currently available in the market in fact have limited bitterness-blocking effects. For example, most known bitter-reducing compounds are not able to reduce caffeine bitterness completely, with masking effects usually remaining below 50%, for example neodiosmine, poly-gamma-glutamic acid, cellotrioside, homoeriodictyol, eriodictyol, gamma-amino butyric acid, alpha-alpha-trehalose, taurine, L-theanine, 2,4-dihydroxybenzoic acid, 2-4-dihydroxybenzoic acid N-vanillyl amide, [2]-gingerdione.

Accordingly, there remains a need in the art for alternative or improved bitterness-blocking compounds and compositions.

### SUMMARY OF THE INVENTION

The present invention addresses the problems described above by providing novel bitter blockers.

In one aspect, the present invention relates to a method of reducing or blocking the bitter taste of an orally consumable composition that includes one or more bitter-tasting substances (bitter tastants), where the method involves adding to the orally consumable product an effective amount of a bitter Mocker selected from the group consisting of eriodictyol-8-C-β-glucoside, homoeriodictyol 4′-O-glucoside, and homoeriodictyol 7-O-glucoside.

For example, the one or more bitter tastants can be selected from the group consisting of caffeine, bitter methylxanthines, theobromine, rebaudioside A, a B vitamin, cannabidiol, tetrahydrocannabinol, nicotine, dextromethorphan, dextromethorphan hydrobromide, chlorhexidine, guaifenesin, pseudoephedrine, atorvastatin, aspirin, acetaminophen, diphenhydramine, doxylamine, sildenafil citrate, and loperamide. In various embodiments, the orally consumable composition can include a high concentration of bitter tastants. For example, the orally consumable composition can include at least 100 mg/L, at least 250 mg/L, at least 500 mg/L, at least 750 mg/L, at least 1,000 mg/L, at least 5000 mg/L, at least 10,000 mg/L, or at least 20,000 mg/L of the one or more bitter tastants.

After screening many flavonoids and flavonoid glycosides, the inventors have surprisingly discovered that eriodictyol-8-C-β-glucoside, homoeriodictyol 4′-O-glucoside, arid homoeriodictyol 7-O-glucoside have superior bitterness-blocking properties. Specifically, by sensory evaluations, it was found that each of eriodictyol-8-C-β-glucoside, homoeriodictyol 4′-O-glucoside, and homoeriodictyol 7-O-glucoside, even at very low concentrations (e.g., between about 10 ppm and about 200 ppm), can reduce by at least 50% (e.g., at least 50%, at least 60%, at least 70%, at least 80%, at least 90%, at least 95%, or at least 99%) of the bitter taste of an orally consumable composition that includes at least 100 mg/L of bitter tastants. In some embodiments, the bitter taste is reduced by at least 60%. In certain embodiments, the bitter taste is reduced by at least 80%. In preferred embodiments, the bitter taste is reduced by 100%.

Accordingly, in another aspect, the present teachings provide an orally consumable composition that includes a) one or more bitter instants, and b) a bitter blocker selected from the group consisting of eriodictyol-8-C-β-glucoside, homoeriodictyol 4′-O-glucoside, and homoeriodictyol 7-O-glucoside. Because of the surprising effectiveness of the bitter blockers of the present invention, the bitter blocker can be present in the orally consumable composition at a very low concentration even if the orally consumable composition has a high concentration of bitter tastants. For example, the orally consumable composition can include at least 100 mg/L, at least 250 mg/L, at least 500 mg/L, at least 750 mg/L, at least 1,000 mg/L, at least 5000 mg/L, at least 10,000 mg/L, or at least 20,000 mg/L of the one or more bitter tastants. Meanwhile, the bitter blocker can be present in a concentration between about 10 ppm and about 200 ppm.

The orally consumable composition can be a food product, a functional food, a beverage product, a pharmaceutical, a dietary supplement, a nutraceutical, a dental hygiene composition, a food grade gel composition, a cosmetic product, and a flavoring product.

Non-exhaustive examples of food products can include cereal products, rice products, tapioca products, sago products, baker's products, biscuits, bread, breakfast cereal, cereal bar, energy bars/nutritional bars, granola, cakes, cookies, crackers, donuts, muffins, pastries, chocolates, ices, honey products, treacle products, yeast products, baking-powder, salt products, spice products, savory products, mustard products, vinegar products, sauces (condiments), tobacco products, cigars, cigarettes, processed foods, cooked fruits, vegetable products, meat, meat products, jellies, jams, gelatins, fruit sauces, egg products, milk products, dairy products, yoghurts, cheese products, butter, butter substitute products, milk substitute products, soy products, edible oils, fat products, food extracts, plant extracts, meat extracts, and condiments. A functional food can be any of the foregoing food products with dietary supplements or nutraceuticals added.

Non-exhaustive examples of beverage products can include coffee, tea, fermented tea, a dairy beverage, a plant-based milk beverage, an alcoholic beverage, flavored water, vitamin water, fruit juice, and an energy drink.

A dietary supplement can include compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

A nutraceutical can include any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.), Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

A gel can refer to any colloidal systems in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

A composition including one of the present bitter blockers can include various pharmaceuticals known in the art. In. certain embodiments, a pharmaceutical composition of the present disclosure can contain from about 5 ppm to about 200 ppm of the present bitter blocker, and one or more pharmaceutically acceptable excipients. In some embodiments, pharmaceutical compositions of the present disclosure can be used to formulate pharmaceutical drugs containing one or more active agents that exert a biological effect. Accordingly, in some embodiments, pharmaceutical compositions of the present disclosure can contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA.

The present bitter blocker also can be used with any suitable dental and oral hygiene compositions known in the art. Examples of suitable dental and oral hygiene compositions can be, for example, toothpastes, tooth polishes, dental floss, mouthwashes, mouthrinses, dentrifices, mouth sprays, mouth refreshers, plaque rinses, dental pain relievers, and the like.

Further provided herein are uses of a bitter blocker selected from the group consisting of eriodictyol-8-C-β-glucoside, homoeriodictyol 4′-O-glucoside, and homoeriodictyol 7-O-glucoside, for reducing or blocking the bitter taste of one or more bitter tastants.

In some embodiments, the one or more bitter tastants are selected from the group consisting of: caffeine, bitter methylxanthines, theobromine, rebaudioside A, a B vitamin, cannabidiol, tetrahydrocannabinol, nicotine, dextromethorphan, dextromethorphan hydrobromide, chlorhexidine, guaifenesin, pseudoephedrine, atorvastatin, aspirin, acetaminophen, diphenhydramine, doxylamine, sildenafil citrate, and loperamide.

In some embodiments, the one or more hitter tastants are in an orally consumable composition. In some embodiments, the bitter blocker reduces the bitter taste of the orally consumable composition by at least 50% (e.g., at least 50%, at least 60%, at least 70%, at least 80%, at least 90%, at least 95%, or at least 99%).

Also provided herein are methods of preparing a flavonoid glycoside, the method comprising incubating a reaction mixture comprising: a) uridine diphosphate-glucose, eriodictyol as a substrate, and c) a glycosyltransferase comprising an amino acid sequence having at least 70% (e.g., at least 70%, at least 80%, at least 90%, at least 95%, at least 99%, or 100%) sequence identity to SEQ ID NO: 1, wherein a glucose is covalently coupled to the eriodictyol substrate to produce eriodictyol-8-C-β-glucoside, optionally wherein the glycosyltransferase comprises the amino acid sequence of SEQ ID NO: 1.

Also provided herein are methods of preparing a flavonoid glycoside, the method comprising incubating a reaction mixture comprising: a) uridine diphosphate-glucose, b) homoeriodictyol as a substrate, and c) a glycosyltransferase comprising an amino acid sequence having at least 70% (e.g., at least 70%, at least 80%, at least 90%, at least 95%, at least 99%, or 100%) sequence identity to SEQ ID NO: 3 or SEQ II) NO: 5, wherein a glucose is covalently coupled to the homoeriodictyol substrate to produce homoeriodictyol 4′-O-glucoside and/or homoeriodictyol 7-O-glucoside, optionally wherein the glycosyltransferase comprises the amino acid sequence of SEQ ID NO: 3 or SEQ ID NO: 5.

In some embodiments, the reaction mixture is in vitro. In some embodiments, the reaction mixture is a cell-based reaction mixture. In some embodiments, the cell-based reaction mixture comprises a cell comprising a polynucleotide encoding the glycosyltransferase. In some embodiments, the cell is a bacterial cell. In some embodiments, the cell is an Escherichia coli (E. coli) cell.

Host cells that comprise a polynucleotide encoding a glycosyltransferase, wherein the polynucleotide comprises a nucleotide sequence that is at least 70% at least 70%, at least 80%, at least 90%, at least 95%, at least 99%, or 100%) identical to any one of SEQ NOs: 2, 4, 6, are provided in some aspects. In some embodiments, the polynucleotide comprises the sequence of any one of SEQ ID NOs: 2, 4, 6. In some embodiments, the host cell is a bacterial cell. In some embodiments, the host cell is an Escherichia coli (E. coli) cell.

Further provided herein are reaction mixtures comprising:

(a) uridine diphosphate-glucose,

(b) a natural flavanone, and

(c) a host cell comprising a glycosyltransferase comprising an amino acid sequence having at least 70% (e.g., at least 70%, at least 80%, at least 90%, at least 95%, at least 99%, or 100%) sequence identity to any one of SEQ ID NOs: 1, 3, 5.

In some embodiments, the natural flavanone is homoeriodictyol, eriodictyol, or combinations thereof. In some embodiments, the host cell is a bacterial cell. In some embodiments, the host cell is an Escherichia coli (E. coli) cell. In sonic embodiments, the glycosyltransferase comprises an amino acid sequence of any one of SEQ ID NOs: 1, 3, 5. In some embodiments, the reaction mixture further comprises: eriodictyol-8-C-β-glucoside, homoeriodictyol 4′-O-glucoside, homoeriodictyol 7-O-glucoside, or combinations thereof.

Compounds produced by the method described herein are provided. Further provided herein are compounds selected from eriodictyol-8-C-β-glucoside, homoeriodictyol 4′-O-glucoside, and homoeriodictyol 7-O-glucoside, and compositions comprising such compounds.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings if present.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 shows the results of ID and 2D NMR analyses of bitter blocker candidate 09 (BB09).

FIG. 2 shows the chemical structure of BB09.

FIG. 3 shows the results of HPLC analysis of BB09 standard (top panel) and purified BB09 (bottom panel).

FIG. 4 shows the results of 1D and 2D NMR analyses of bitter blocker candidate 11 (BB11).

FIG. 5 shows the chemical structure of BB11.

FIG. 6 shows the results of HPLC analysis of BB 11 standard (top panel) and purified BB11 (bottom panel).

FIG. 7 shows the results of H-NMR analysis of bitter blocker candidate 13 (BB13) in deuterated dimethyl sulfoxide (DMSO-d6).

FIG. 8 shows the results of H-NMR analysis of BB13 in DMSO with d6-D₂O-exchange.

FIG. 9 shows the chemical structure of BB13.

FIG. 10 shows the results of HPLC analysis of BB13 standard (top panel) and purified BB13 (bottom panel).

FIG. 11 shows the results of a two-alternative forced choice (2AFC) difference test completed by fifteen panelists, Each panelist provided two separate evaluations, resulting in a total of thirty evaluations. Results are statistically significant at the 90% and 95% confidence interval.

FIG. 12 shows a concentration-responsive curve of Compound A (BB09), Compound B (BB11), and Compound C (BB13) with 100 μM Dextromethorphan-HBr as the Bitter Stimulus. Response was measured by luminescence, *p<0.05 by one-way ANOVA.

FIG. 13 shows a concentration-responsive curve of Compound A (BB09), Compound B (BB11), Compound C (BB13), Senomyx BB68, and STX001 with 400 μM L-Praziquantel in pooled donor-derived human taste bud tissue-derived cells (hTBEC). Response was measured by luminescence. *p<0.05 by one-way ANOVA.

FIGS. 14A-14B show normalized concentration-responsive curves of Compound A (BB09), Compound B (BB11), Compound C (BB13), STX001, Sodium Gluconate, Eridyctiol, Homoeridictyol, and Semonyx B868 with either 100 μM Dextromethorphan-HBr stimulus in individual donor-derived hTBECs (FIG. 14A) or 400 μM L-Praziquantel stimulus in individual donor-derived hTBECs (FIG. 14B). Response was measured by luminescence. *p<0.05 by one-way ANOVA.

FIG. 15 shows real time ATP secretion in hTBEC 66 in response to 300 μM Theobromine alone (Vehicle), 300 μM Theobromine with 1000 μM of Senomyx BB68, or 300 μM Theobromine with 1000 μM of Compound C (BB13).

FIG. 16 shows ATP secretion signal in pooled hTBEC 56 cultures in response to a DMSO control, 3mM Rebaudioside A with Compound A (BB09), 3 mM Rebaudioside A with Compound B (BB11), 3 mM Rebaudioside A with Compound C (BB13), 3 mM Rebaudioside A with Senomyx BB68, 3 mM Rebaudioside A with ST X001, 3 mM Rebaudioside A with Homoeridictyol, 3 mM Rebaudioside A with Eridictyol, and 3 mM Rebaudioside A with Sodium Gluconate. Each antagonist treated with 3 mM Rebaudioside A is provided as DMSO control, 100 μM, 300 μM, or 1,000 μM. *p<0.05 by one-way ANOVA.

FIG. 17 shows real time ATP secretion in pooled hTBEC 56 cultures in response to 1 mM Rebaudioside A alone (Vehicle), 1 mM Rebaudioside A with 1,000 μM Compound A (BB09), 1 mM Rebaudioside A with 1,000 μM Compound C (BB13), and 1 mM Rebaudioside A with 1,000 μM Senomyx BB68.

FIGS. 18A-18C show real time ATP secretion detection profiling of three hTBEC donor cultures: hTBEC 66 (FIG. 18A), hTBEC 56 (FIG. 18B), and hTBEC Donor H (FIG. 18C). Each donor culture was treated with 100 μM Dextromethorphan-HBr alone (Vehicle), 100 μM Dextromethorphan-HBr with 100 μM Compound A (BB09), 100 μM Dextromethorphan-HBr with 100 μM Compound B (BB11), 1.00 μM Dextromethorphan-HBr with 100μM Compound C (BB13), 100 μM Dextromethorphan-HBr with 100 μM STX001, or 100 μM Dextromethorphan-HBr with 100 μM Senomyx BB68. *p<0.05 by one-way ANOVA.

FIGS. 19A-19C show real time ATP secretion detection profiling of three hTBEC donor cultures: hTBEC 66 (FIG. 19A), hTBEC 56 (FIG. 19B), and hTBEC Donor H (FIG. 19C). Each donor culture was treated with 1,000 μM Theobromine alone (Vehicle), 1,000 μM Theobromine with 1,000 μM Compound A (BB09), 1,000 μM Theobromine with 1,000 μM Compound B (BB11), 1,000 μM Theobromine with 1,000 μM Compound C (BB13), 1,000 μM Theobromine with 1,000 μM STX001, or 1,000 μM Theobromine with 1,000 μM Senomyx BB68. *p<0.05 by one-way ANOVA.

FIGS. 20A-20C show real time ATP secretion detection profiling of three hTBEC donor cultures: hTBEC 66 (FIG. 20A), hTBEC 56 (FIG. 20B), and hTBEC Donor H (FIG. 20C). Each donor culture was treated with 1 mM Rebaudioside A alone (Vehicle), 1 mM Rebaudioside A with 1 mM Compound A (BB09), 1 mM Rebaudioside A with 1 mM Compound B (BB11), 1 mM Rebaudioside A with 1 mM Compound C (BB13), 1 mM Rebaudioside A with 1 mM STX001, or 1 mM Rebaudioside A with 1 mM Senomyx BB68. *p<0.05 by one-way ANOVA.

FIGS. 21A-21C show real time ATP secretion detection profiling of three hTBEC donor cultures: hTBEC 66 (FIG. 21A), hTBEC 56 (FIG. 21B), and hTBEC Donor H (FIG. 21C). Each donor culture was treated with 3 mM Caffeine alone (Vehicle), 3 mM Caffeine with 3 mM Compound A (BB09), 3 mM Caffeine with 3 mM Compound B (BB11), 3 mM Caffeine with 3 mM Compound C (BB13), 3 mM Caffeine with 3 mM STX001, or 3 mM Caffeine with 3 mM Senomyx BB68. *p<0.05 by one-way ANOVA.

FIGS. 22A-22B show ATP secretion detection profiling of three hTBEC donor cultures: hTBEC 66, hTBEC 56, and hTBEC Donor H. FIG. 22A shows the response of each donor culture to either 100 μM Dextromethorphan-HBr alone (Vehicle) or 100 μM Dextromethorphan-HBr with 1 mM Compound C (BB13). FIG. 22B shows the response of each donor culture to either 1,000 μM Theobromine alone (Vehicle) or 1,000 μM Theobromine with 1 mM Compound C (BB13). *p<0.05 by one-way ANOVA.

FIGS. 23A-23B show ATP secretion detection profiling of three hTBEC donor cultures: hTBEC 66, hTBEC 56, and hTBEC Donor H. FIG. 23A shows the response of each donor culture to either 1 mM Rebaudioside A alone (Vehicle) or 1 mM Rebaudioside A with 1 mM Compound C (BB13). FIG. 23B shows the response of each donor culture to either 3 ,M Caffeine alone (Vehicle) or 3 mM Caffeine with 1 mM Compound C (BB13). *p<0.05 by one-way ANOVA.

FIG. 24 shows ATP secretion detection profiling of three hTBEC donor cultures: hTBEC 15 66, hTBEC 56, and hTBEC Donor H. Each culture was treated with either 400 mM L-Praziquantel alone (Vehicle) or 400 mM L-Praziquantel with 1 mM Compound C (BB13). *p<0.05 by one-way ANOVA.

FIG. 25 shows a cell calcium mobilization assay from individual donor-derived hTBECs in response to treatment with 100 μM Dextromethorphan-HBr and Compound C (BB13) at a concentration from 0.3 μM to 1,000 μM.

FIG. 26 shows a cell calcium mobilization assay from individual donor-derived hTBECs in response to treatment with 300 μM Theobromine and Compound C (BB13) at a concentration from 1 μM to 3,000 μM.

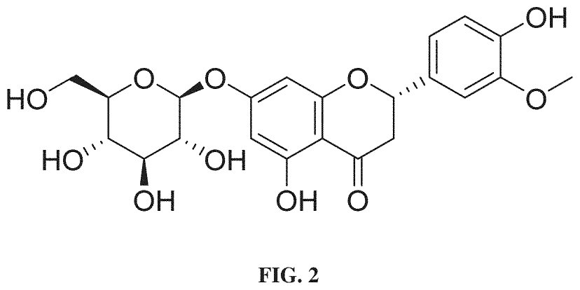

FIG. 2 shows a cell calcium mobilization assay from hTBEC 56 cells in response to treatment with 3 mM Caffeine alone (Vehicle), 3 mM Caffeine with 1,000 μL Senomyx BB68, or 3 mM Caffeine with 1,000 μM Compound C (BB13).

### DETAILED DESCRIPTION

Bitter-tasting substances or bitter tastants within the meaning of the present disclosure can be, for example, xanthine alkaloids (e.g., caffeine, theobromine), bitter methylxanthines pyridine alkaloids (e.g., nicotine), quinoline derivatives (e.g., quinine), limonoids limonine from citrus fruits), polyphenols catechols, flavonols, gamma-oryzanol, hesperitin), pharmaceutically active compounds (e.g., fluoroquinoline antibiotics, aspirin, beta-lactam antibiotics, ambroxol, paracetamol, aspirin, guaifenesin), dextromethorphan, dextromethorphan hydrobromide, rebaudioside A, denatonium benzoate, sucralose octaacetate, potassium chloride, magnesium salts, urea, bitter amino acids (e.g., tryptophan), and bitter peptide fragments (e.g., having a terminal leucine or isoleucine radical). As shown in the examples below, bitter blockers according to the present invention are extremely effective in reducing or blocking the bitter taste originated from various bitter tastants.

The present bitter blockers also can be effective in reducing or blocking a bitter off-taste or aftertaste. Substances which have a bitter aftertaste within the meaning of the present disclosure can be, for example, an artificial or a natural sweetener with a bitter aftertaste selected from the group consisting of abiziasaponin, abrusosides (e.g., abrusoside A, abrusoside B, abrusoside C, abrusoside D), acesulfame potassium, advantame, albiziasaponin, alitame, aspartame, superaspartame, bayunosides (e.g., bayunoside 1, bayunoside 2) brazzein, bryoside, bryonoside, bryonodulcoside, carnosifloside, carrelame, curculin, cyanin, chlorogenic acid, cyclamates and its salts, cyclocarvoside I, dihydroquercetin-3-acetate, dihydroflavenol, dulcoside, gaudichaudioside, glycyrrhizin, glycyrrhetin acid, gypenoside, hematoxylin, hernandulcin, isomogrosides (e.g., iso-mogroside V), lugduname, magap, mabinlins, micraculin, mogrosides (e.g., mogroside IV and mogroside V), monatin and its derivatives, monellin, mukurozioside, naringin dihydrochalcone (NarDHC), neohesperidin dihydrochalcone (NDHC), neotame, osladin, pentadin, periandrin I-V, perillartine, D-phenylalanine, phlomsosides, in particular phlomisoside 1, phlomisoside 2, phlomisoside 3, phlomisoside 4, phloridzin, phyllodulcin, polpodiosides, polypodoside A, pterocaryosides, rebaudiosides (e.g., rebaudioside A, rebaudioside B, rebaudioside C, rebaudioside D, rebaudioside F, rebaudioside G, rebaudioside H), rubusosides, saccharin and its salts and derivatives, scandenoside, selligueanin A, siamenosides (e.g., siamenoside I), strogines (e.g., strogin 1, strogin 2, strogin 4), suavioside A, suavioside B, suavioside G, suavioside H, suavioside I, suavioside J, sucralose, sucronate, sucrooctate, talin, telosmoside A15, thaumatin (e.g., thaumatin I and II), trans-anethol, trans-cinnamaldehyde, trilobatin, and D-tryptophane, including extracts or enriched fractions of the natural sweeteners.

In some embodiments, the present bitter blockers, i.e., eriodictyol-8-C-β-glucoside, homoeriodictyol 4′-O-glucoside, and/or homoeriodictyol 7-O-glucoside, are selected for their ability to reduce the bitterness of certain bitter tastants, and yet not completely block the desired bitter notes typical of, for example, coffee and chocolate, or their aroma.

In various embodiments, the present invention relates to methods of using eriodictyol-8-C-β-glucoside, homoeriodictyol 4′-O-glucoside, and/or homoeriodictyol 7-O-glucoside as hitter blockers. The method generally includes adding to a consumable composition comprising a bitter tastant an amount of at least one of the present bitter blockers that is effective to modify, mask, reduce and/or suppress the hitter taste of the bitter tastant, wherein the amount of the bitter blocker can be less than a taste threshold concentration associated with the bitter blocker, and wherein the effect of the bitter blocker remains at least as long as the taste of the bitter tastant is perceived. In the context of the present invention, the term “threshold” concentration means that the hitter blocker is present in an amount at which it is either not recognizable and/or identifiable and/or does not exert an undesired taste effect, but still exerts its respective bitter blocking effects.

In some embodiments, the consumable composition can include a sweetener that provides a complimentary masking effect to the bitter-blocking effect of the bitter blocker. In other embodiments, the consumable composition can exclude sweeteners.

In certain embodiments, the consumable composition can include a flavor agent. The flavor agent can be chosen from synthetic flavor oils and flavoring aromatics, and/or oils, oleo resins and extracts derived from plants, leaves, flowers, fruits and so forth, and combinations thereof. Representative flavor oils include cinnamon oil, peppermint oil, clove oil, bay oil, eucalyptus oil, thyme oil, cedar leaf oil, oil of nutmeg, oil of sage, and oil of bitter almonds. Also useful are artificial, natural or synthetic fruit flavors such as vanilla, and citrus oil, including lemon, orange, grape, lime and grapefruit and fruit essences including apple, pear, peach, strawberry, raspberry, cherry, plum, pineapple, apricot and so forth. Any of these flavor agents may be used individually or in admixture. Commonly used flavors include mints such as peppermint, menthol, vanilla, cinnamon derivatives, and various fruit flavors, whether employed individually or in admixture. Flavor agents such as aldehydes and esters including cinnamyl acetate, cinnamaldehyde, citral, diethyllacetal, dihydrocarvyl acetate, eugenyl formate, p-methylanisole, and so forth may also be used. Generally, any flavoring or food additive such as those described in Chemicals Used in Food Processing, pub 1274 by the National Academy of Sciences, pages 63-258 may be used as flavor agents in the invention.

In general, the consumable compositions of the present invention can be prepared utilizing techniques well known to those of ordinary skill in the art. As such, the consumable compositions of the present invention may include various other components which are customarily used in the preparation of such consumable compositions, and which would be known to those of skill in the art.

The present consumable composition can be formulated into various forms including tablets, chews, edible films, gels, solutions, suspensions, emulsions, and so forth. For example, when the consumable composition of the present invention is in the form of a liquid pharmaceutical composition, or even a toothpaste, dental cream, or gel, such a form typically includes a liquid carrier material for the bitter tastant and the bitter blocker. The carrier material may comprise water, typically in an amount of from about 10% to about 90% by weight of the consumable composition. Carrier materials include, but are not limited to, polyethylene glycol (PEG), propylene glycol (PG), glycerin or mixtures thereof. In addition, the consumable composition may include humectants, such as, for example, sorbitol, glycerin, and polyalcohols. Particularly advantageous liquid ingredients comprise mixtures of water with polyethylene glycol, propylene glycol, or glycerin and sorbitol. A gelling agent (thickening agent) including natural or synthetic gums, such as sodium carboxymethylcellulose, hydroxyethyl cellulose, methyl cellulose and the like, may also be used, typically in the range of about 0.15% to about 1.30% by weight of the consumable composition. In a toothpaste, dental cream or gel, the liquids and solids are proportioned to form a creamy or gelled mass which is extrudable from a pressurized container or from a collapsible tube.

The consumable composition of the present invention may also include a thickening agent or binder. For example, the thickening agent or binder may be selected from the group consisting of finely particulate gel silicas and nonionic hydrocolloids, such as carboxymethyl cellulose, sodium hydroxymethyl cellulose, hydroxyethylcellulose, hydroxypropyl guar, hydroxyethyl starch, polyvinyl pyrrolidone, vegetable gums, such as tragacanth, agar, carrageenans, gum arabic, xanthan gum, guar gum, locust bean gum, carboxyvinyl polymers, fumed silica, silica clays and the like, and combinations thereof. For example, a preferred thickening agent for use in toothpastes is carrageenan available under the trade names GELCARIN® and VISCARIN® from FMC Biopolymers, Philadelphia, Pa., U.S.A. Other thickening agents or binders are polyvinyl pyrrolidone available from Noveon, Inc. Cleveland, Ohio, U.S.A. wider the trademark CARBOPOL®, fumed silica under the trademark CAB-O-SIL® available from Cabot Corporation, Boston, Mass., U.S.A., and silica clays available from Laporte industries, Ltd., London, U.K. under the trademark LAPOINTE®, The thickening agent or binder may be used with or without a carrier; such as glycerol, polyethylene glycol (e.g., PEG-400), or combinations thereof; however, when a carrier is used; preferably up to about 5% thickening agent or hinder, more preferably from about 0.1% to about 1.0%, is combined with preferably from about 95.0% to about 99.9% carrier, more preferably from about 99.0% to about 99.9%, based on the total weight of the thickening agent/carrier combination. Furthermore, when the thickening agent or binder is a hydrated silica and it is used with a carrier, preferably from about 5% to about 10% thickening agent or binder is combined with preferably from about 90% to about 95% carrier, based on the total weight of the thickening agent/carrier combination.

The consumable composition of the present invention may also contain coloring agents or colorants, such as colors, dyes, pigments, and particulate substances, in amounts effective to produce the desired color of the particular consumable composition. The coloring agents (colorants) useful in the invention include the pigments such as titanium dioxide, which may be incorporated in amounts of up to about 2% by weight of the consumable composition, and preferably less than about 1% by weight. Colorants may also include natural food colors and dyes suitable for food, drug and cosmetic applications. For example, food grade and/or pharmaceutically acceptable coloring agents, dyes, or colorants, as would be understood to one 20 skilled in the art, include FD&C colorants such as primary FD&C Blue No. 1, FD&C Blue No. 2, FD&C Green No. 3, FD&C Yellow No. 5, FD&C Yellow No. 6, FD&C Red No. 3, FD&C Red No. 33 and FD&C Red No. 40 and lakes FD&C Blue No. 1; FD&C Blue No. 2, FD&C Yellow No. 5, FD&C Yellow No. 6, FD&C Red No. 2, FD&C Red No. 3, FD&C Red No. 33, FD&C Red No. 40 and combinations thereof.

In addition, the consumable composition of the invention may also include a surfactant, such as sodium lauryl sulfate (SLS) (preferably in an amount of from about 1% to about 2% of the total weight of the oral composition), and/or a preservative, such as sodium benzoate (preferably in an amount of about 0.2% of the total weight of the oral composition).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Bitter Blocker Candidates

Table 1 below provides the chemical name, formula, molar mass, and chemical structure of various bitter blocker candidates.

TABLE 1

| Sample | | | Molar | |
|---|---|---|---|---|
| Name | Chemical Name | Formula | Mass | Chemical Structure |
| BB01 | Acacetin | C₁₆H₁₂O₅ | 284.26 g/mol | 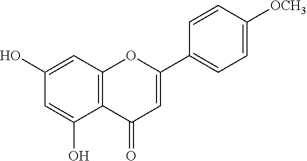 [SMILES: COc1ccc(-c2cc(=O)c3c(O)cc(O)cc3o2)cc1] |
| BB02 | Homoeriodictyol | C₁₆H₁₄O₆ | 302.2788 g/mol | 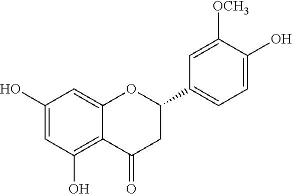 [SMILES: COc1cc([C@@H]2CC(=O)c3c(O)cc(O)cc3O2)ccc1O] |
| BB03 | Kaempferol | C₁₅H₁₀O₆ | 286.23 g/mol | 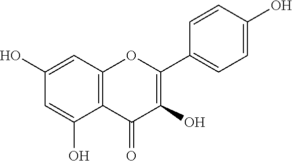 |
| BB04 | Dihydroquercetin | C₁₅H₁₂O₇ | 304.25 g/mol | 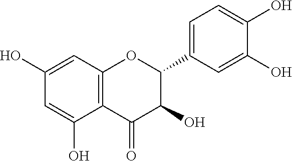 [SMILES: O=C1c2c(O)cc(O)cc2O[C@H](c2ccc(O)c(O)c2)[C@H]1O] |
| BB05 | Dihydroquercetin glucoside | C₂₁H₂₀O₁₂ | 466.0955 g/mol | 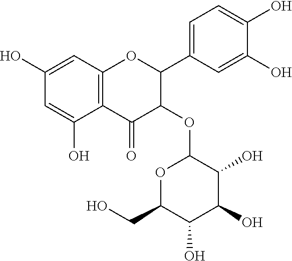 [SMILES: O=C1c2c(O)cc(O)cc2OC(c2ccc(O)c(O)c2)C1OC1O[C@H](CO)[C@@H](O)[C@H](O)[C@H]1O] |
| BB06 | Dihydrokaempferol | C₁₅H₁₂O₆ | 288.25 g/mol | 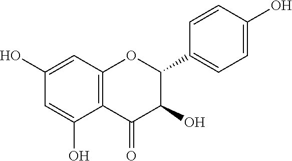 [SMILES: O=C1c2c(O)cc(O)cc2O[C@H](c2ccc(O)cc2)[C@H]1O] |
| BB07 | Eriodictyol | C₁₅H₁₂O₆ | 288.25 g/mol | 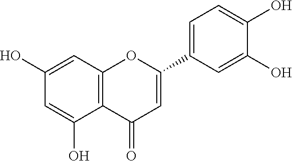 |
| BB08 | Dibydromyricetin | C₁₅H₁₂O₈ | 320.25 g/mol | 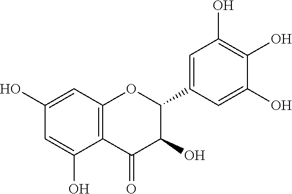 [SMILES: O=C1c2c(O)cc(O)cc2O[C@H](c2cc(O)c(O)c(O)c2)[C@H]1O] |
| BB09 | Homoeriodictyol 7- O-glucoside | C₂₂H₂₄O₁₁ | 464.4 g/mol | 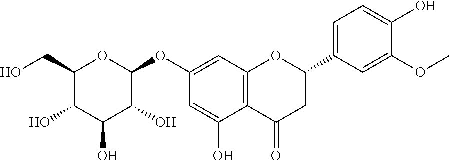 [SMILES: COc1cc([C@@H]2CC(=O)c3c(O)cc(O[C@@H]4O[C@H](CO)[C@@H](O)[C@H](O)[C@H]4O)cc3O2)ccc1O] |
| BB10 | Eriodictyol 7-O- glucoside | C₂₁H₂₂O₁₁ | 450.39 g/mol | 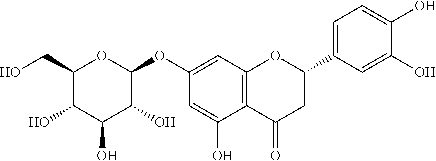 [SMILES: O=C1C[C@@H](c2ccc(O)c(O)c2)Oc2cc(O[C@@H]3O[C@H](CO)[C@@H](O)[C@H](O)[C@H]3O)cc(O)c21] |
| BB11 | Homoeriodictyol 4′-O-glucoside | C₂₂H₂₄O₁₁ | 464.4 g/mol | 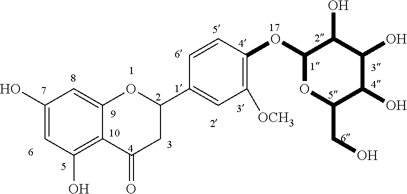 |
| BB12 | Eriodictyol 4′-O- glucoside | C₂₁H₂₂O₁₁ | 450.39 g/mol | 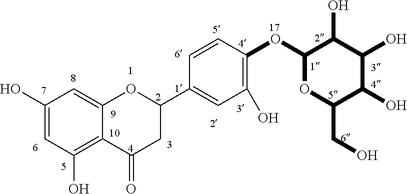 |
| BB13 | Eriodictyol-8-C-β- glucoside | C₂₁H₂₂O₁₁ | 450.39 g/mol | 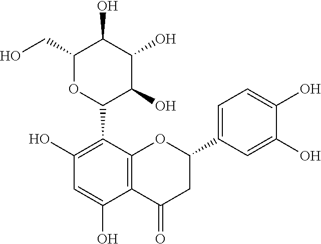 |

### Example 1—Reducing the Bitterness of a Caffeine Solution

The bitter blocker candidates listed in Table 1 were prepared into a 1% sample solution with propylene glycol (i.e., 1 g of bitter blocker per 100 ml of propylene glycol) and heated to ensure complete solubilization. Each of the sample solutions were observed to give a slightly yellow color appearance. Individually, the respective sample solution was added to a 0.25% caffeine solution (i.e., a concentration of 0.25 g caffeine in 100 ml of water), which by itself tasted bitter on all areas of the tongue. A trained sensory evaluator was asked to estimate the perceived bitterness reduction against the control, as well as to provide comments on how the addition of the individual sample solution modulated the taste and mouth feel profile of the resulting caffeine solution. The results are summarized in Table 2 below.

TABLE 2

| | | Estimated % | |
|---|---|---|---|
| Sample | Dosage | bitterness | |
| Name | (ppm) | reduction | Comments |
| BB01 | 20 | 20% | Some bitterness blocking on sides |
| | | | and tip of tongue |
| BB02 | 20 | 30% | Some blocking on sides and tip of |
| | | | tongue |
| BB03 | 20 | 20% | Some bitterness blocking on sides |
| | | | and tip of tongue |
| BB04 | 30 | 40% | Some bitterness blocking on sides |
| | | | and tip of tongue |
| BB05 | 30 | 20% | Some bitterness blocking on the back |
| | | | of the tongue, minimal reduction |
| BB06 | 50 | 40% | Some bitterness blocking on sides |
| | | | and back of tongue |
| BB07 | 50 | 50% | Some bitterness blocking on all parts |
| | | | of the tongue, good reduction |
| BB08 | 50 | 20% | Some bitterness blocking on front of |
| | | | the tongue, minimal reduction |
| BB09 | 50-100 | 60-80% | Good upfront bitterness reduction |
| | | | with significant bitterness reduction |
| BB10 | 50-100 | 30% | Some bitterness blocking on front of |
| | | | the tongue, minimal reduction |
| BB11 | 50-100 | 60-80% | Blocks all parts of the tongue with |
| | | | significant bitterness reduction |
| BB12 | 50-100 | 30% | Some bitterness blocking on front |
| | | | and sides of the tongue, minimal |
| | | | reduction |
| BB13 | 50-100 | 80-100% | Blocks all parts of the tongue with |
| | | | almost total bitterness blocking |

### Example 2—Reducing the Bitterness of a Peach-Flavored Energy Drink

The bitterness blocker candidates listed in Table 1 were prepared into a 1% sample solution with propylene glycol and heated to ensure complete solubilization. Each of the sample solutions were observed to give a slightly yellow color appearance. Individually, the respective sample solution was added to a commercially available peach-flavored energy drink which has a bitter taste due to the presence of caffeine (168 mg/8 fl oz serving or about 710 mg/l) and the recommended daily allowances of various B vitamins. A trained sensory evaluator was asked to estimate the perceived bitterness reduction against the control, as well as to provide comments on how the addition of the individual sample solution modulated the taste and mouth feel profile of the resulting peach-flavored energy drink. The results are summarized in Table 3 below.

TABLE 3

| | | Estimated % | |
|---|---|---|---|
| Sample | Dosage | bitterness | |
| Name | (ppm) | reduction | Comments |
| BB01 | 20 | 20% | Some bitterness blocking on |
| | | | sides and tip of tongue |
| BB02 | 20 | 30% | Some blocking on sides and tip |
| | | | of tongue |
| BB03 | 20 | 20% | Some bitterness blocking on |
| | | | sides and tip of tongue |
| BB04 | 20 | 40% | Some bitterness blocking on |
| | | | sides and tip of tongue |
| BB05 | 20 | 20% | Some bitterness blocking on the |
| | | | back of the tongue |
| BB06 | 50 | 40% | Some bitterness blocking on |
| | | | sides and back of tongue |
| BB07 | 50 | 50% | Some bitterness blocking on all |
| | | | parts of the tongue |
| BB08 | 50 | 20% | Some bitterness blocking on |
| | | | front of the tongue |
| BB09 | 50-100 | 60-80% | Good upfront bitterness |
| | | | reduction |
| BB10 | 50-100 | 30% | Some bitterness blocking on |
| | | | front of the tongue |
| BB11 | 50-100 | 80% | Good bitterness reduction with |
| | | | just a slight bitterness in the end |
| BB12 | 50-100 | 30% | Some bitterness blocking on |
| | | | front and sides of the tongue |
| BB13 | 50-100 | 80-100% | Excellent bitter blocking |
| | | | throughout |

### Example 3—Reducing the Bitterness of Dark Chocolate Pieces

The bitterness blocker candidates listed in Table 1 were prepared into a 1% sample solution with propylene glycol and heated to ensure complete solubilization. Each of the sample solutions were observed to give a slightly yellow color appearance. Individually, the respective sample solution was added to melted dark chocolate pieces with 100% dark cacao. A trained sensory evaluator was asked to estimate the perceived bitterness reduction against the control, as well as to provide comments on how the addition of the individual sample solution modulated the taste and mouth feel profile of the resulting melted dark chocolate pieces. The results are summarized in Table 4 below.

TABLE 4

| | | Estimated % | |
|---|---|---|---|
| Sample | Dosage | bitterness | |
| Name | (ppm) | reduction | Comments |
| BB01 | 50 | 20% | Some bitterness blocking on |
| | | | sides and tip of tongue |
| BB02 | 50 | 20% | Some blocking on sides and tip |
| | | | of tongue |
| BB03 | 50 | 20% | Some bitterness blocking on |
| | | | sides and tip of tongue |
| BB04 | 50 | 40% | Some bitterness blocking on |
| | | | sides and tip of tongue |
| BB05 | 50 | 20% | Some bitterness blocking on the |
| | | | back of the tongue |
| BB06 | 50 | 40% | Some bitterness blocking on |
| | | | sides and back of tongue |
| BB07 | 50 | 50% | Some bitterness blocking on all |
| | | | parts of the tongue |
| BB08 | 50 | 20% | Some bitterness blocking on |
| | | | front of the tongue |
| BB09 | 100 | 40-60% | Good bitterness reduction but |
| | | | bitterness still there |
| BB10 | 100 | 30% | Some bitterness blocking on |
| | | | front of the tongue |
| BB11 | 100 | 60% | Good bitterness reduction but |
| | | | bitterness still there |
| BB12 | 100 | 30% | Some bitterness blocking on |
| | | | front and sides of the tongue |
| BB13 | 100 | 80-100% | Complete bitter blocking with |
| | | | good upfront finish |

### Example 4—Reducing the Bitterness of Dark Roast Coffee

The bitterness blocker candidates listed in Table 1 were prepared into a 1% sample solution with propylene glycol and heated to ensure complete solubilization. Each of the sample solutions were observed to give a slightly yellow color appearance. Individually, the respective sample solution was added to dark roast coffee (which is estimated to contain about 175 mg of caffeine per 8 fl oz, or about 740 mg/l)). A trained sensory evaluator was asked to estimate the perceived bitterness reduction against the control, as well as to provide comments on how the addition of the individual sample solution modulated the taste and mouth feel profile of the resulting dark roast coffee. The results are summarized in Table 5 below.

TABLE 5

| | | Estimated % | |
|---|---|---|---|
| Sample | Dosage | bitterness | |
| Name | (ppm) | reduction | Comments |
| BB01 | 50 | 20% | Some bitterness blocking on |
| | | | sides and tip of tongue |
| BB02 | 50 | 20% | Some blocking on sides and tip |
| | | | of tongue |
| BB03 | 50 | 20% | Some bitterness blocking on |
| | | | sides and tip of tongue |
| BB04 | 50 | 40% | Some bitterness blocking on |
| | | | sides and tip of tongue |
| BB05 | 50 | 20% | Some bitterness blocking on the |
| | | | back of the tongue |
| BB06 | 50 | 40% | Some bitterness blocking on |
| | | | sides and back of tongue |
| BB07 | 50 | 50% | Some bitterness blocking on all |
| | | | parts of the tongue |
| BB08 | 50 | 20% | Some bitterness blocking on |
| | | | front of the tongue |
| BB09 | 50 | 60-80% | Nice upfront bitterness reduction |
| | | | with some bitter finish in the end |
| BB10 | 100 | 30% | Some bitterness blocking on |
| | | | front of the tongue |
| BB11 | 100 | 80% | Nice upfront bitterness reduction |
| | | | with some bitter finish in the end |
| BB12 | 100 | 30% | Some bitterness blocking on |
| | | | front and sides of the tongue |
| BB13 | 100 | 80-100% | Very smooth with nice upfront |
| | | | bitter blocking as well as nice |
| | | | finish in the end. |

### Example 5—Reducing the Bitterness of Cough Syrup

The bitterness blocker candidates listed in Table 1 were prepared into a 1% sample solution with propylene glycol and heated to ensure complete solubilization. Each of the sample solutions were observed to give a slightly yellow color appearance. Individually, the respective sample solution was added to a cough syrup sold under the trade name DELSYM®. The bitter-tasting agent contained in the cough syrup were dextromethorphan HBr USP 30 mg (as measured for each 5 ml teaspoon, i.e., 6000 mg/l of dextromethorphan). A trained sensory evaluator was asked to estimate the perceived bitterness reduction against the control, as well as to provide comments on how the addition of the individual sample solution modulated the taste and mouth feel profile of the resulting cough syrup. The results are summarized in Table 6 below.

TABLE 6

| | | Estimated % | |
|---|---|---|---|
| Sample | Dosage | bitterness | |
| Name | (ppm) | reduction | Comments |
| BB01 | 50 | 20% | Some bitterness blocking on |
| | | | sides and tip of tongue |
| BB02 | 50 | 20% | Some blocking on sides and tip |
| | | | of tongue |
| BB03 | 50 | 20% | Some bitterness blocking on |
| | | | sides and tip of tongue |
| BB04 | 50 | 20% | Some bitterness blocking on |
| | | | sides and tip of tongue |
| BB05 | 50 | 20% | Some bitterness blocking on the |
| | | | back of the tongue |
| BB06 | 50 | 40% | Some bitterness blocking on |
| | | | sides and back of tongue |
| BB07 | 50 | 30% | Some bitterness blocking on all |
| | | | parts of the tongue |
| BB08 | 50 | 20% | Some bitterness blocking on |
| | | | front of the tongue |
| BB09 | 100 | 50% | Covered up a good percentage of |
| | | | bitterness. |
| BB10 | 100 | 30% | Some bitterness blocking on |
| | | | front of the tongue |
| BB11 | 100 | 60% | Less bitter, smoother, more |
| | | | palatable, sweeter in taste. |
| BB12 | 100 | 30% | Some bitterness blocking on |
| | | | front and sides of the tongue |
| BB13 | 100 | 80% | Almost blocked all the |
| | | | bitterness, sweeter, more |
| | | | palatable, thicker in mouth feel. |

Example 6—Reducing the Bitterness of Cough Syrup

The bitterness blocker candidates listed in Table 1 were prepared into a 1% sample solution with propylene glycol and heated to ensure complete solubilization. Each of the sample solutions were observed to give a slightly yellow color appearance. Individually, the respective sample solution was added to a cough syrup sold under the trade name ROBITUSSIN® DM. The bitter-tasting agents contained in the cough syrup were dextromethorphan HBr USP 20 mg and guaifenesin USP 400 mg (as measured for each 20 ml serving, or 21000 mg/l of bitter tastants in total). A trained sensory evaluator was asked to estimate the perceived bitterness reduction against the control, as well as to provide comments on how the addition of the individual sample solution modulated the taste and mouth feel profile of the resulting cough syrup. The results are summarized in Table 7 below.

TABLE 7

| | | Estimated % | |
|---|---|---|---|
| Sample | Dosage | bitterness | |
| Name | (ppm) | reduction | Comments |
| BB01 | 50 | 20% | Some bitterness blocking on |
| | | | sides and tip of tongue |
| BB02 | 50 | 20% | Some blocking on sides and tip |
| | | | of tongue |
| BB03 | 50 | 20% | Some bitterness blocking on |
| | | | sides and tip of tongue |
| BB04 | 50 | 20% | Some bitterness blocking on |
| | | | sides and tip of tongue |
| BB05 | 50 | 20% | Some bitterness blocking on the |
| | | | back of the tongue |
| BB06 | 50 | 40% | Some bitterness blocking on |
| | | | sides and back of tongue |
| BB07 | 50 | 30% | Some bitterness blocking on all |
| | | | parts of the tongue |
| BB08 | 50 | 20% | Some bitterness blocking on |
| | | | front of the tongue |
| BB09 | 100 | 50% | Covered up a good percentage of |
| | | | bitterness. |
| BB10 | 100 | 30% | Some bitterness blocking on |
| | | | front of the tongue |
| BB11 | 100 | 60% | Less bitter, smoother, more |
| | | | palatable, sweeter in taste. |
| BB12 | 100 | 30% | Some bitterness blocking on |
| | | | front and sides of the tongue |
| BB13 | 100 | 80% | Almost blocked all the |
| | | | bitterness, sweeter, more |
| | | | palatable. |

### Example 7—Reducing the Bitterness of Full Spectrum CBD Hemp Oil

The bitterness blocker candidates listed in Table 1 were prepared into a 1% sample solution with propylene glycol and heated to ensure complete solubilization. Each of the sample solutions were observed to give a slightly yellow color appearance. A full spectrum CBD hemp oil was emulsified into a water-soluble nanoemulsion first, to which was added the respective sample solution. The full spectrum CBD hemp oil by itself has an earthy, musky, bitter taste. A trained sensory evaluator was asked to estimate the perceived bitterness reduction against the control, as well as to provide comments on how the addition of the individual sample solution modulated the taste and mouth feel profile of the resulting oil nanoemulsion. The results are summarized in Table 8 below.

TABLE 8

| | | Estimated % | |
|---|---|---|---|
| Sample | Dosage | bitterness | |
| Name | (ppm) | reduction | Comments |
| BB01 | 50 | 30% | Some bitterness blocking on |
| | | | sides and tip of tongue |
| BB02 | 50 | 30% | Some blocking on sides and tip |
| | | | of tongue |
| BB03 | 50 | 30% | Some bitterness blocking on |
| | | | sides and tip of tongue |
| BB04 | 50 | 30% | Some bitterness blocking on |
| | | | sides and tip of tongue |
| BB05 | 50 | 30% | Some bitterness blocking on the |
| | | | back of the tongue |
| BB06 | 50 | 40% | Some bitterness blocking on |
| | | | sides and back of tongue |
| BB07 | 50 | 30% | Some bitterness blocking on all |
| | | | parts of the tongue |
| BB08 | 50 | 30% | Some bitterness blocking on |
| | | | front of the tongue |
| BB09 | 100 | 60% | Nice upfront bitterness reduction |
| | | | with some bitter finish in the end |
| BB10 | 100 | 30% | Some bitterness blocking on |
| | | | front of the tongue |
| BB11 | 100 | 60% | Nice upfront bitterness reduction |
| | | | with slight bitterness in the end |
| BB12 | 100 | 30% | Some bitterness blocking on |
| | | | front and sides of the tongue |
| BB13 | 100 | 80% | Almost blocked all the |
| | | | bitterness, very smooth finish. |

### Example 8—Reducing the Bitterness of CBD Isolate

The bitterness blocker candidates listed in Table 1 were prepared into a 1% sample solution with propylene glycol and heated to ensure complete solubilization. Each of the sample solutions were observed to give a slightly yellow color appearance. A CBD isolate was emulsified into a water-soluble nanoemulsion first, to which was added the respective sample solution. The CBD isolate by itself was noted to have an earthy flavor. A trained sensory evaluator was asked to estimate the perceived bitterness reduction against the control, as well as to provide comments on how the addition of the individual sample solution modulated the taste and mouth feel profile of the resulting nanoemulsion. The results are summarized in Table 9 below.

TABLE 9

| | | Estimated % | |
|---|---|---|---|
| Sample | Dosage | bitterness | |
| Name | (ppm) | reduction | Comments |
| BB01 | 50 | 30% | Some bitterness blocking on |
| | | | sides and tip of tongue |
| BB02 | 50 | 30% | Some blocking on sides and tip |
| | | | of tongue |
| BB03 | 50 | 30% | Some bitterness blocking on |
| | | | sides and tip of tongue |
| BB04 | 50 | 30% | Some bitterness blocking on |
| | | | sides and tip of tongue |
| BB05 | 50 | 30% | Some bitterness blocking on the |
| | | | back of the tongue |
| BB06 | 50 | 40% | Some bitterness blocking on |
| | | | sides and back of tongue |
| BB07 | 50 | 30% | Some bitterness blocking on all |
| | | | parts of the tongue |
| BB08 | 50 | 30% | Some bitterness blocking on |
| | | | front of the tongue |
| BB09 | 100 | 60% | Nice upfront bitterness reduction |
| | | | with some bitter finish in the end |
| BB10 | 100 | 30% | Some bitterness blocking on |
| | | | front of the tongue |
| BB11 | 100 | 60% | Nice upfront bitterness reduction |
| | | | with slight bitterness in the end |
| BB12 | 100 | 30% | Some bitterness blocking on |
| | | | front and sides of the tongue |
| BB13 | 100 | 80% | Almost blocked all the |
| | | | bitterness, very smooth finish. |

### Example 9—Reducing the Bitterness of THC

The bitterness blocker candidates listed in Table 1 were prepared into a 1% sample solution with propylene glycol and heated to ensure complete solubilization. Each of the sample solutions were observed to give a slightly yellow color appearance. THC was first emulsified into a water-soluble nanoemulsion (10 mg THC per serving), to which was added the respective sample solution. A trained sensory evaluator was asked to estimate the perceived bitterness reduction against the control, as well as to provide comments on how the addition of the individual sample solution modulated the taste and mouth feel profile of the resulting nanoemulsion. The results are summarized in Table 10 below.

TABLE 10

| | | Estimated % | |
|---|---|---|---|
| Sample | Dosage | bitterness | |
| Name | (ppm) | reduction | Comments |
| BB01 | 50 | 30% | Some bitterness blocking on |
| | | | sides and tip of tongue |
| BB02 | 50 | 30% | Some blocking on sides and tip |
| | | | of tongue |
| BB03 | 50 | 30% | Some bitterness blocking on |
| | | | sides and tip of tongue |
| BB04 | 50 | 30% | Some bitterness blocking on |
| | | | sides and tip of tongue |
| BB05 | 50 | 30% | Some bitterness blocking on the |
| | | | back of the tongue |
| BB06 | 50 | 40% | Some bitterness blocking on |
| | | | sides and back of tongue |
| BB07 | 50 | 30% | Some bitterness blocking on all |
| | | | parts of the tongue |
| BB08 | 50 | 30% | Some bitterness blocking on |
| | | | front of the tongue |
| BB09 | 100 | 60% | Nice upfront bitterness reduction |
| | | | with some bitter finish in the end |
| BB10 | 100 | 30% | Some bitterness blocking on |
| | | | front of the tongue |
| BB11 | 100 | 60% | Nice upfront bitterness reduction |
| | | | with slight bitterness in the end |
| BB12 | 100 | 30% | Some bitterness blocking on |
| | | | front and sides of the tongue |
| BB13 | 100 | 80% | Almost blocked all the |
| | | | bitterness, very smooth finish. |

### Example 10—Biosynthesis of Flavonoid Glycosides

Different glycosyltransferases were identified for the preparation of flavonoid glycosides of interest. Specifically, TcCGT1, a putative flavone 8-C-glycosyltransferase from the transcriptome (BioProject accession number PRJNA532685) of Trollius chinensis, described in e of al., “Molecular Characterization and Structural Basis of a Promiscuous C-Glycosyltransferase from Trollius chinensis,” Angew. Chem. Int. Ed., 58(33): 11513-11520 (2019), was used to glycosylate eriodictyol to provide eriodictyol-8-C-β-glucoside (BB013).

UGT73B2 (Arabidopsis gene At4g34135) described in Willits et al., “Bio-fermentation of modified flavonoids: an example of in vivo diversification of secondary metabolites,” Phytochemistry, 65: 31-41 (2004), and BcGT1 from Bacillus cereus (GenBank accession no. AAS41089.1) described in Chiu et al., “Diversity of sugar acceptor of glycosyltransferase 1 from Bacillus cereus and its application for glucoside synthesis,” Appl. Microbiol. Biotechnol., 100: 4459-4471 (2016), were used to glycosylate eriodictyol and homoeriodictyol to provide homoeriodictyol 7-O-glucoside (BB9), and eriodictyol 7-O-glucoside (BB10), homoeriodictyol 4′-O-glucoside (BB11), eriodictyol 4′-O-glucoside (BB12). The protein sequences of TcCGT1, UGT73B2, and BcGT1 are provided as SEQ ID NO: 1, SEQ ID NO: 3, and SEQ ID NO: 5, respectively (Table 11).

TABLE 11

| Protein | Organism | Gene Accession No. | Sequence ID |
|---|---|---|---|
| TcCGT1 | Trollius chinensis | PRJNA532685 | SEQ ID NO: 1 |
| UGT73B2 | Arabidopsis thaliana | At4g34135 | SEQ ID NO: 3 |
| BcGT1 | Bacillus cereus | AAS41089.1 | SEQ ID NO: 5 |

The respective TcCGT1 gene, the UGT73B2 gene, and the BcGT1 gene were cloned into expression vectors, then introduced into E. coli W3110 cells with standard chemical transformation protocol. The resulting E. coli strains carrying the target gene were cultivated under conditions known in the art and stored in glycerol at −70° C. until use.

To produce an E. coli culture suitable for bitter blockier production, glycerol stocks of E. coli W3310 carrying a specific UDP-G glycosyltransferase were removed from −70° C., thawed at room temperature, and cultured in a 50 mL LB culture seed media at 37° C. (termed Seed Culture 1). After 16 hours, Seed Culture 1 was transferred to 2 L of culture seed media, forming Seed Culture 2. Once the cells of Seed Culture 2 produced an OD600 of 5, the cells were transferred to 500 L fermenters, then to a 60 ton production fermenter with modified mineral medium and cultured for 12 hours.

To begin bitter blocker production, either eriodictyol or homoeriodictyol was added to the culture as substrate together with UDP-glucose and the reaction mixture was allowed to incubate for 24 hours. The reaction mixture was then released from the fermenter for down-stream processing.

To extract and purify the bitter blocker products, the reaction mixture was centrifuged and the supernatant was transferred to an ion-exchange resin column. The columns were subsequently washed with warm water and eluted with food-grade ethanol. The eluate was then condensed with a wipe-film condenser. The resulting condensate was transferred to a crystallization tank, crystallized by chilling, re-dissolved in water, passed through activated charcoal to remove any fermentation-based colorant, dried in a baking oven, and crushed into a fine powder for further analyses.

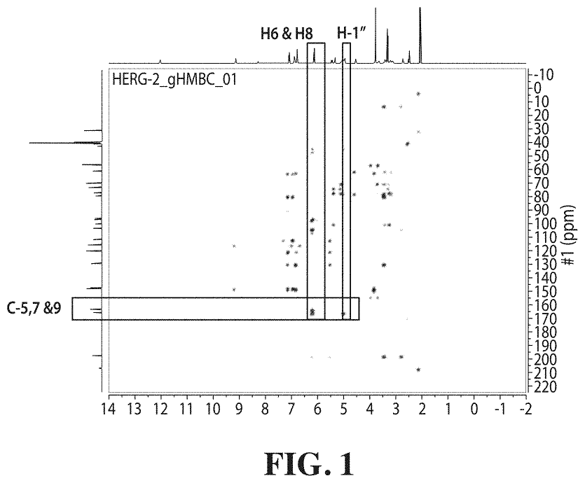

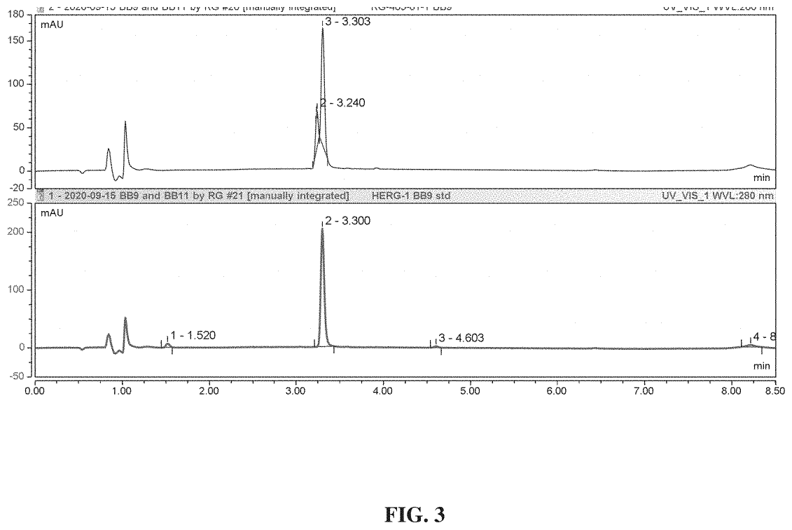

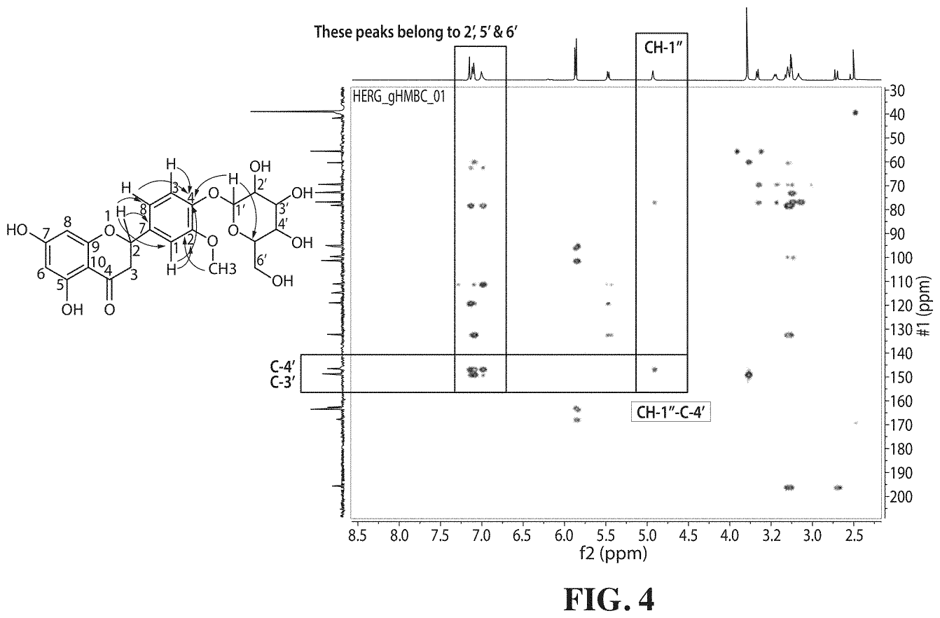

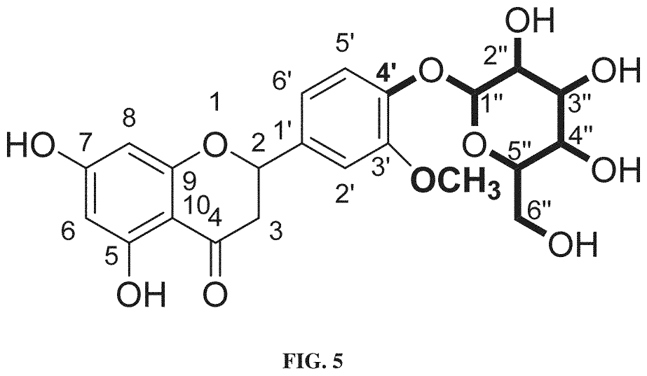

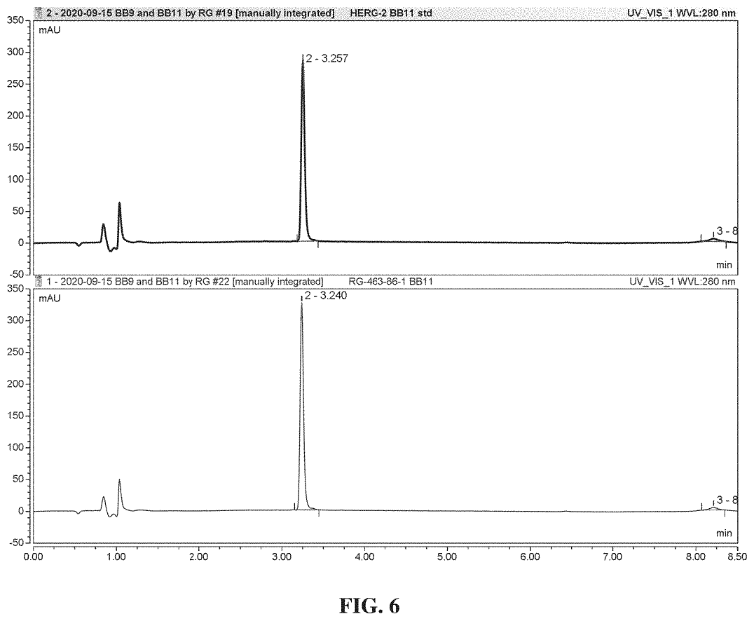

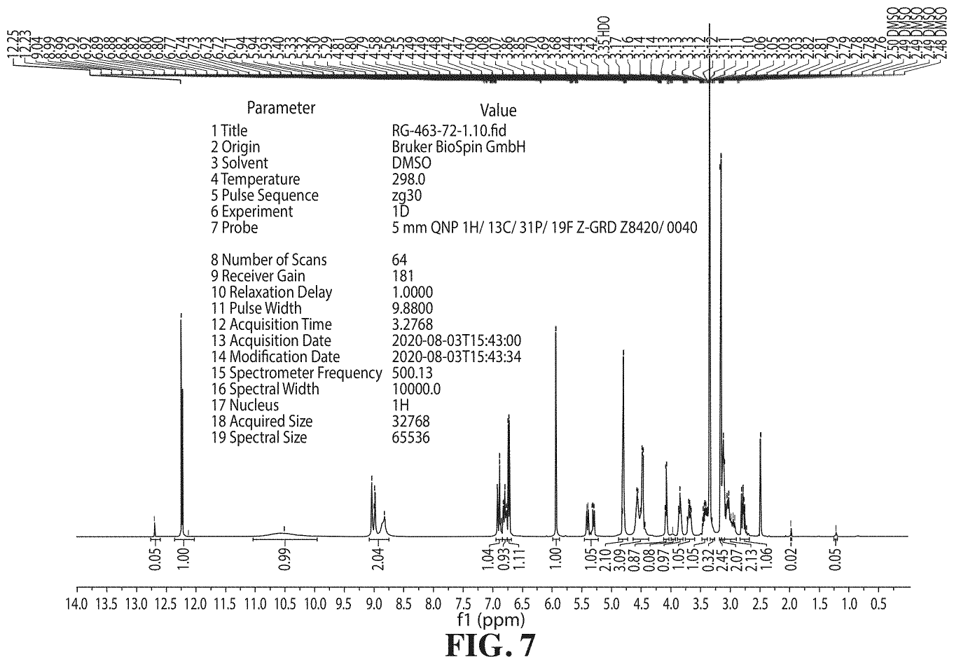

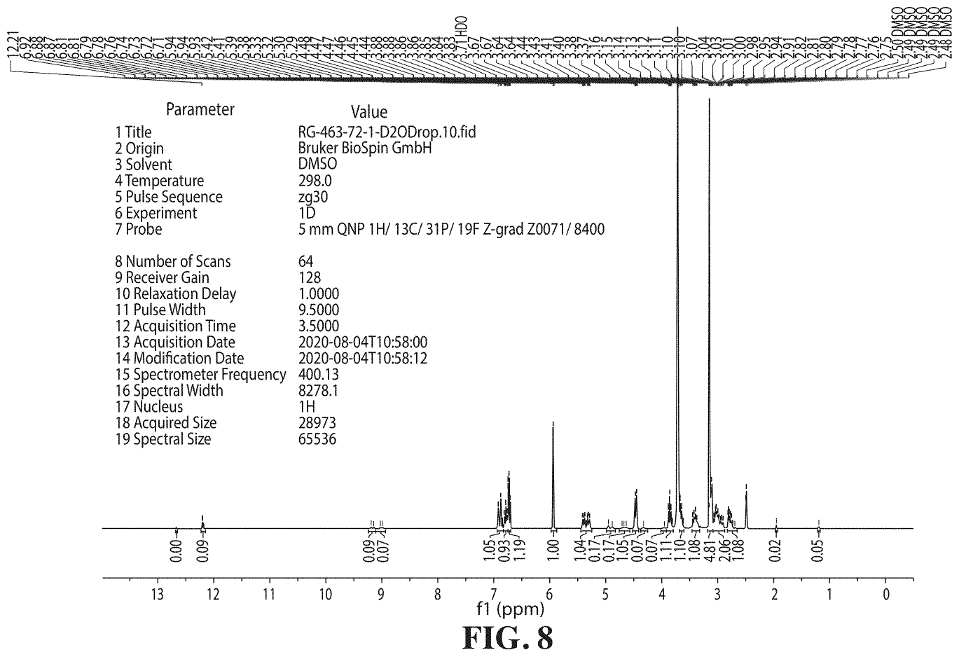

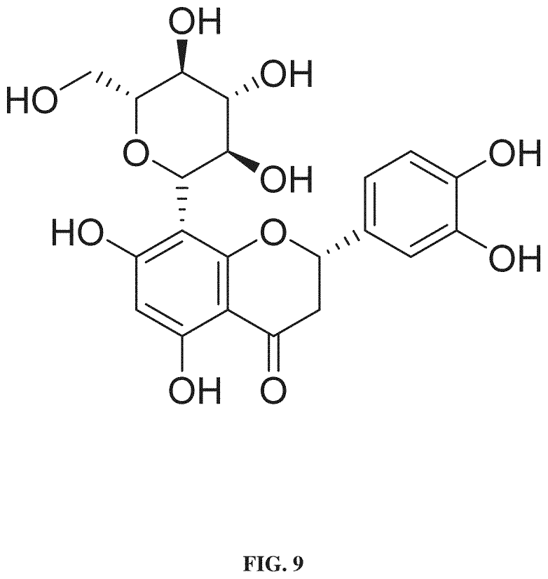

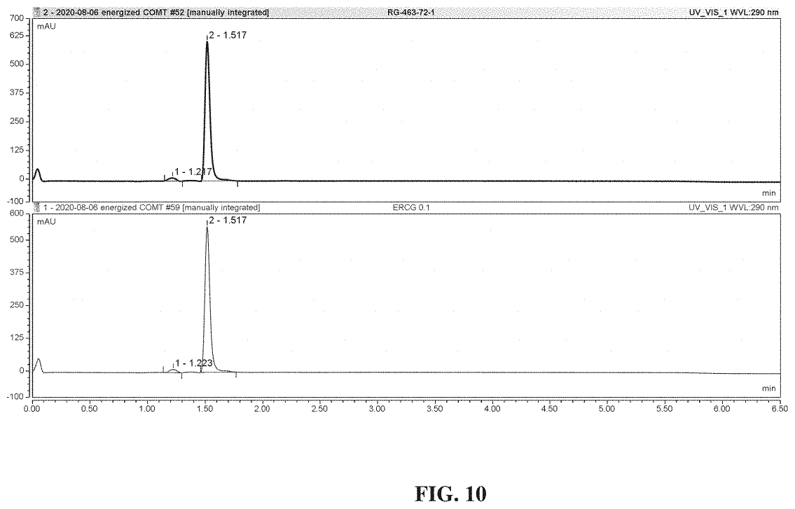

HPLC analysis confirmed production of eriodictyol-8-C-β-glucoside (FIG. 10) from eriodictyol with addition of TcCGT1, production of homoeriodictyol 4′-O-glucoside (FIGS. 3) and homoeriodictyol 7-O-glucoside (FIG. 6) from homoeriodictyol with addition of UGT73B2 or BcGT1, and production of eriodictyol 4′-O-glucoside and eriodictyol 7-O-glucoside from eriodictyol with addition of BcGT1 or UGT73B2. The structure of eriodictyol-8-C-β-glucoside was identified by H-NMR analysis (FIG. 7 and FIG. 8), and the chemical structure is provided in FIG. 9. The structure of homoeriodictyol 4′-O-glucoside was identified by H-NMR (FIG. 1), and the chemical structure is provided in FIG. 2. The structure of homoeriodictyol 7-O-glucoside was identified by H-NMR (FIG. 4), and the chemical structure is provided in FIG. 5.

### Example 11—Reducing Bitterness Using BB09, BB11, and BB13

The bitter blocker candidates BB09, BB11, and BB13 were subject to a two-alternative forced choice (2AFC) difference test wherein panelists were presented Control Caffeine solutions and Test solutions containing caffeine and one of three bitterness blocker candidates (BB09, BB 11, or BB13). Panelists were asked to evaluate the two samples and answer the question, “which one is more hitter?”

BB09 and BB11 were tested first. Three ounces of control (caffeine in water), BB09 in caffeine water, and BB11 in caffeine water were presented at room temperature in separate plastic souffle cups labeled with 3-digit codes. Ten sensory trained panelists evaluated each sample in three repetitions with a 10 minute break between repetitions. Panelists then completed the 2AFC. Data was collected on EveQuestion and analyzed on XLSTAT. Testing was conducted at Sensations Research according to FEMA guidelines.

Control (caffeine in water) and BB09 (BB09 in caffeine water) were significantly different from each other in bitterness at a 90% confidence level, with panelists agreeing that the control sample was more bitter than the sample containing BB09 (Table 12). Of the thirty panelist evaluations collected (10 panelists providing 3 evaluations per Test solution), twenty panelist responses identified the control sample as more bitter, as compared to the sample containing BB09 (P=0.0494, 90% CI).

Control (caffeine in water) and BB11 (BB11 in caffeine water) were significantly different from each other in bitterness at a 90% confidence level, with panelists agreeing that the control sample was more bitter than the sample containing BB11 (Table 12). Of the thirty panelist evaluations collected (10 panelists providing 3 evaluations per Test solution), twenty panelist responses identified the control sample as more bitter, as compared to the sample containing BB11 (P=0.0494, 90% CI).

TABLE 12

| | | | Total | # | # | |
|---|---|---|---|---|---|---|
| | | | Re- | Selected | Selected | |
| Control | Variant | Question? | sponses | Control | Variant | Significant? |
| Caffeine | BB09 | Which is | 30 | 20 | 10 | Yes, |
| in Water | | more bitter? | | | | Control |
| Caffeine | BB11 | Which is | 30 | 20 | 10 | Yes, |
| in Water | | more bitter? | | | | Control |

BB13 was tested separately. Two ounces of control (caffeine in water), BB13 in caffeine water were presented at room temperature in separate plastic souffle cups labeled with 3-digit codes. Fifteen participants evaluated each sample in two repetitions with a 10 minute break between repetitions for a total of thirty evaluations as per the FEMA guidelines. Panelists then completed the 2AFC to identify the more bitter sample. Data was collected and analyzed on Compusense.

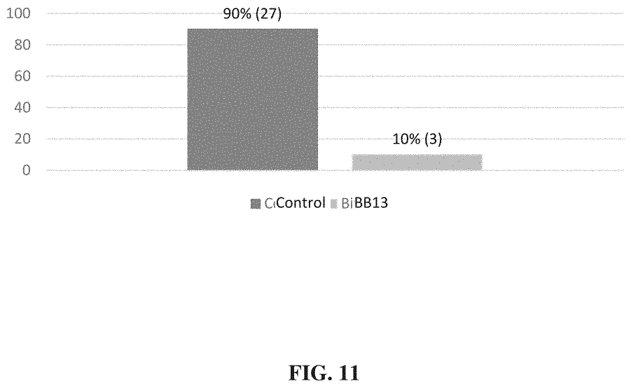

Of the thirty evaluations, three identified the sample containing BB13 as more bitter. Twenty-seven panelists selected control as more bitter. The control sample was significantly more bitter than the sample containing BB13 at the 90% and 95% confidence interval (FIG. 11). Generally, the panelists described samples containing BB13 as having an initial sweetness that mask the bitterness, less lingering bitterness, and having a nutty taste.

### Example 12—Reducing Bitterness of Various Bitter Agonists Using BB09, BB11, and BB13

The bitter blocker candidates were further characterized using bitter-responsive human taste bud tissue-derived cells (hTBEC) platforms and bioassays. Bitter-responsive hTBECs were treated with hitter blocker candidates and various bitter agonists to assess the efficacy of each bitter blocker candidate in reducing the bitterness of each of the various bitter agonists.

Four bitter stimuli were used as bitter agonists (Dextromethorphan-HBr, Caffeine, Theobromine, and Rebaudioside A) and three bitter blocker candidates were assessed (Compound A, or BB09; Compound. B, or BB11; and Compound C, or BB13). One industry standard bitter agonist was used as a control (L-Praziquantel), and five bitter blocker controls were used (Senomyx BB68, STX-001, sodium gluconate, eridictyol, and homoeridictyol).

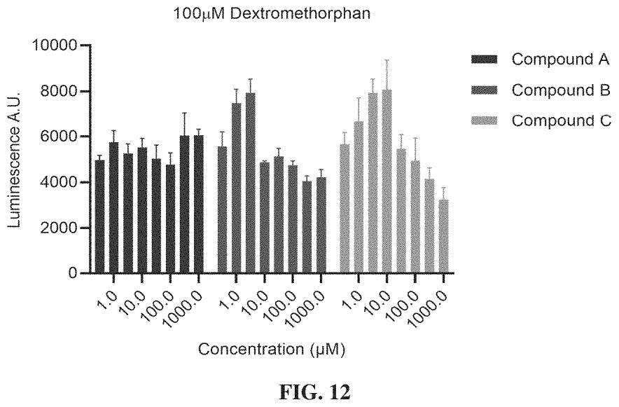

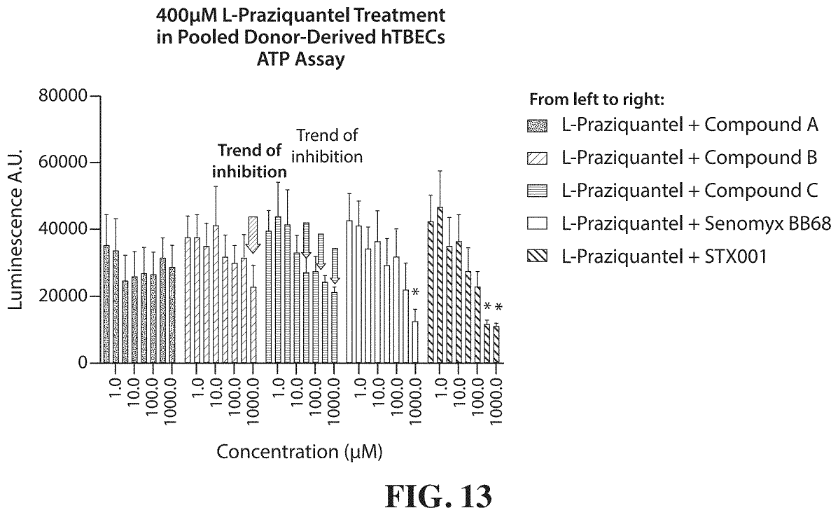

BB09, BB11, and BB13 were first tested at various concentrations in combination with 100 μM Dextromethorphan-HBr. An ATP secretion detection assay was performed to determine whether the bitter blocker candidates were able to inhibit the luminescence activity of Dextromethorphan-HBr. Both BB11 and BB13 were able to inhibit the luminescence activity of Dextromethorphan-HBr (FIG. 12). Similarly, when using L-Praziquantel as an internal control bitterness stimulus, BB11 and BB13, as well as Senomyx BB68 and STX001 showed inhibition of the L-Praziquantel response in the ATP secretion assay (FIG. 13).

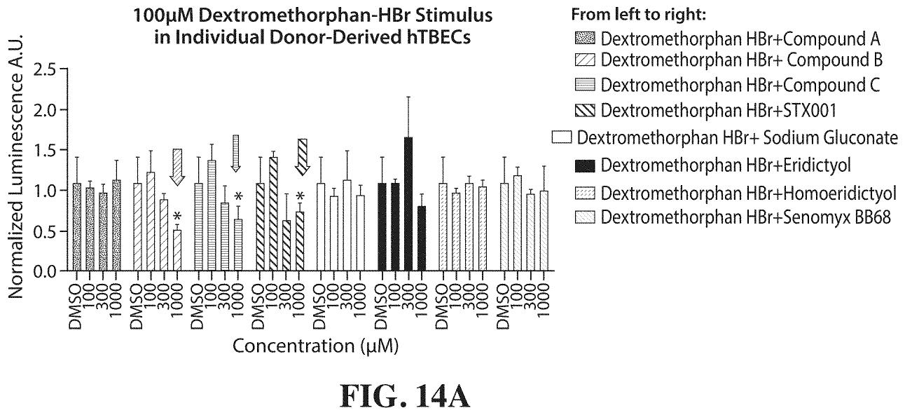

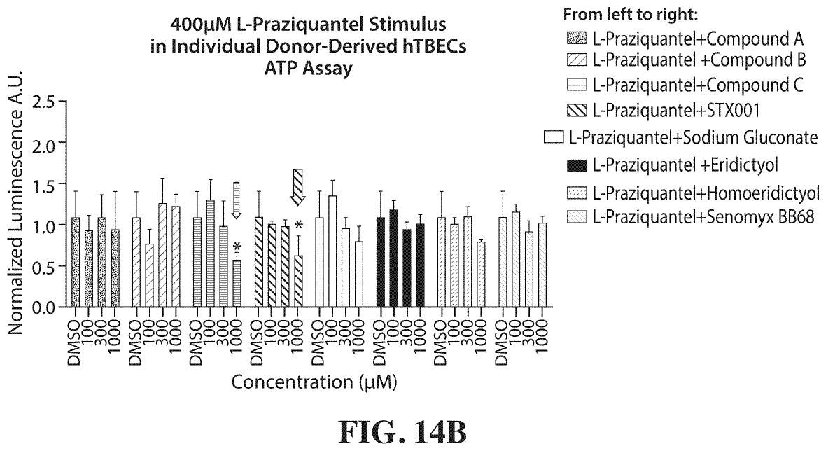

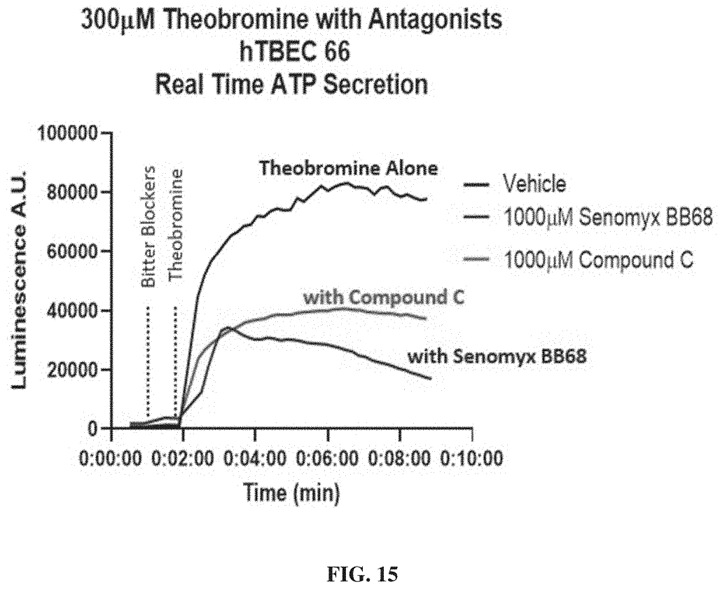

Next, bitter blocker concentration ranges were narrowed (100 μM-1,000 μM) and compared to a fixed concentration of stimulus. Upper concentrations of BB 13 and STX001 inhibited luminescence signal from both 100 μM Dextromethorphan-HBr (FIG. 14A) and 400 μM L-Praziquantel (FIG. 14B). BB13 was evaluated further by real time ATP secretion detection in pooled hTBEC 66 cells treated with 300 μM Theobromine. Both BB 13 and Senomyx BB68 were able to inhibit the ATP secretion response at 1 mM. Theobromine stimulated a rapid increase in ATP secretion of the hTBEC 66 platform that plateaus after 3-4 minutes and begins to decay after 5 minutes. Both BB13 and Senomyx BB68 attenuated the Theobromine-stimulated signal (FIG. 15).

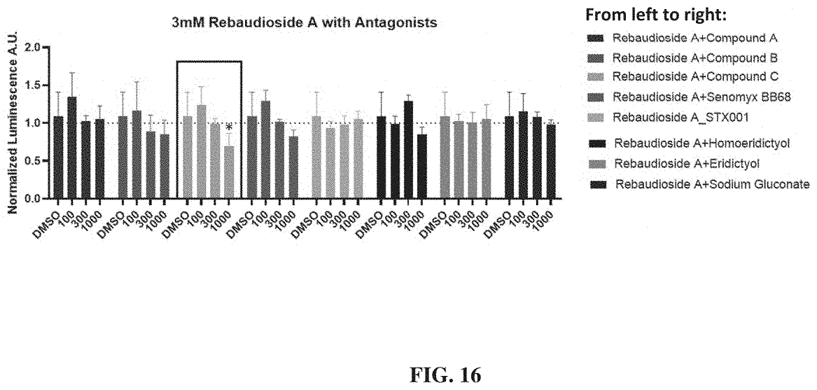

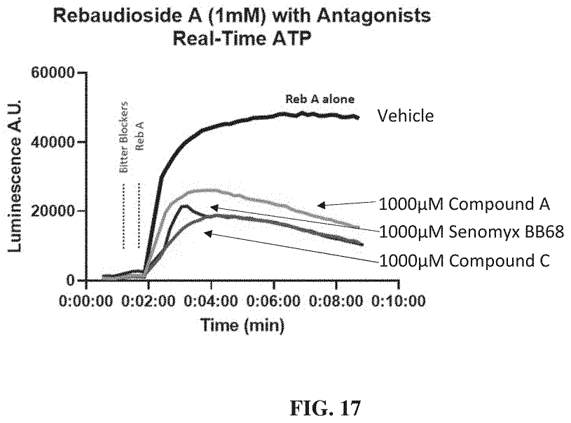

Rebaudioside A elicited an ATP secretion response at a concentration of 3 mM, BB09, BB11, BB13, Senomyx BB68, STX001, Homoeridictyol, Eridictyol, and sodium gluconate were tested with Rebaudioside A in pooled hTBEC 56 cultures. BB13 showed the strongest inhibition of Rebaudioside A-induced ATP secretion (FIG. 16). By real time ATP secretion detection, both BB09 and BB13 attenuated Rebaudioside A-induced ATP secretion in pooled hTBEC 56 cultures (FIG. 17).

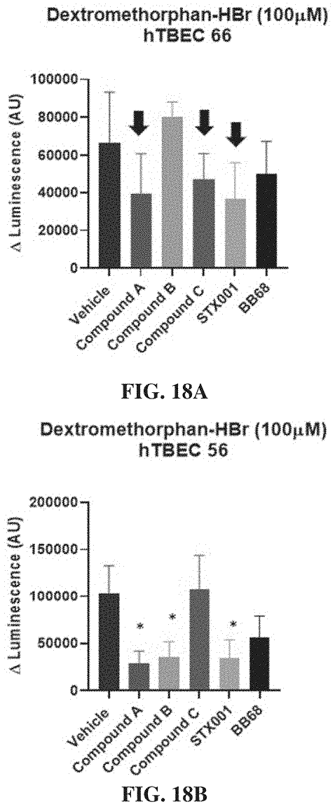

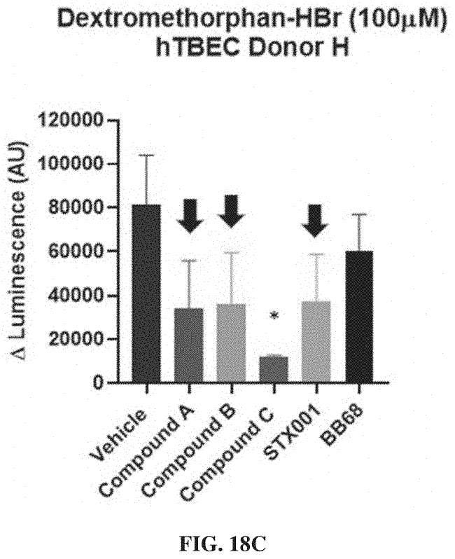

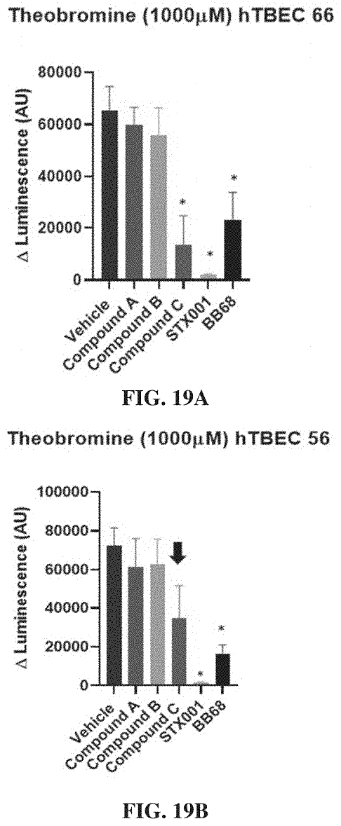

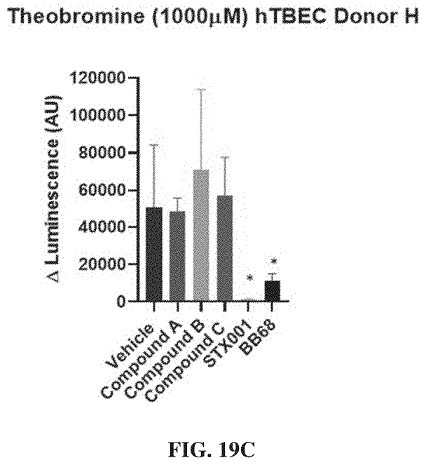

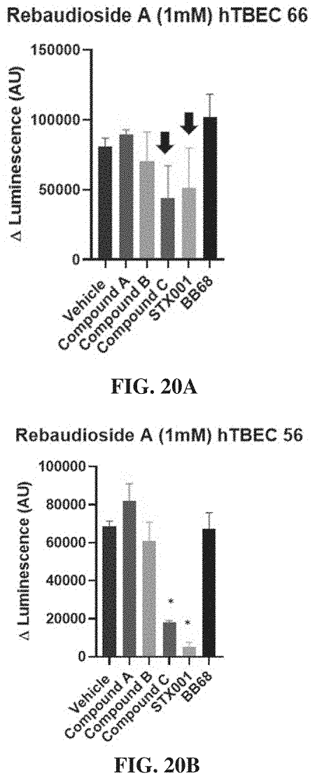

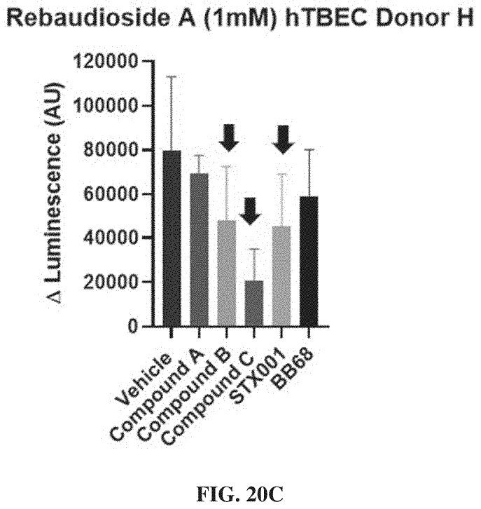

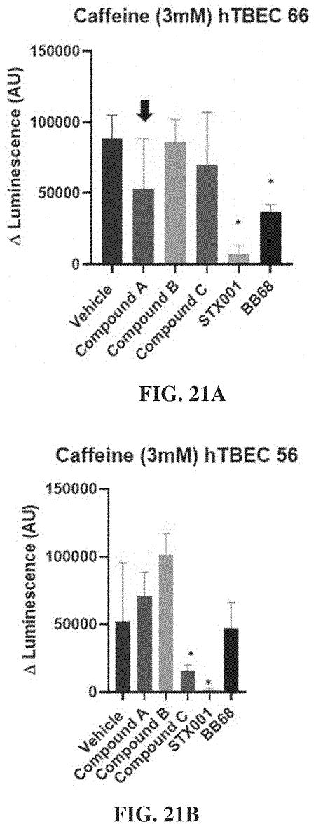

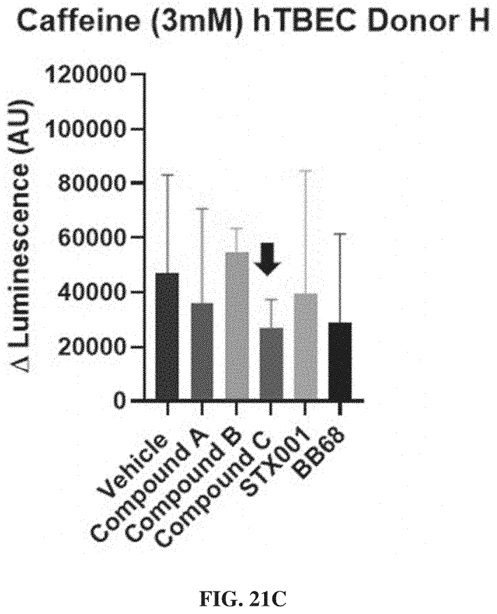

With an understanding of the ideal concentrations for bitter agonists and bitter blocker candidates, an ATP secretion detection assay was performed in three separate hTBEC donor cultures (hTBEC 66, hTBEC 56, and hTBEC Donor H). Each culture was treated with BB09, BB11, BB13, STX001, or Senomyx BB68, as well as either Dextromethorphan-HBr at 1000 μM (FIGS. 18A-18C), Theobromine at 1,000 μM (FIGS. 19A-19C), Rebaudioside A at 1 mM (FIGS. 20A-20C), or Caffeine at 3 mM (FIGS. 21A-21C). BB13 showed the most consistent inhibition of the bitter agonists. BB09 and BB11 each showed trends of inhibition in most cases.

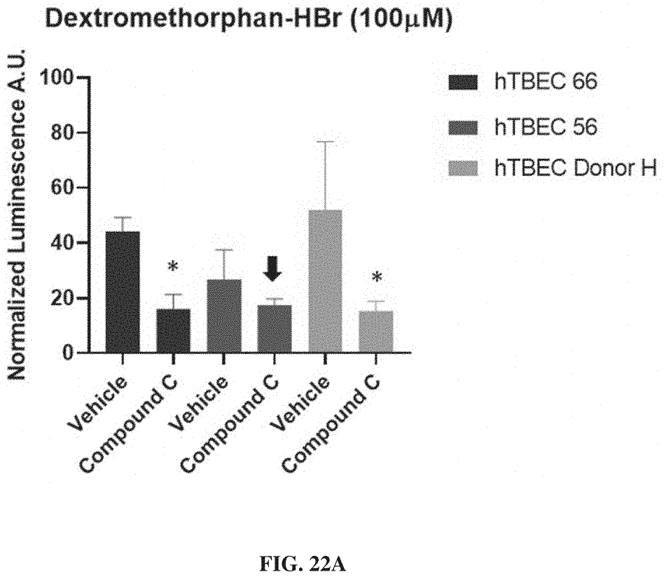

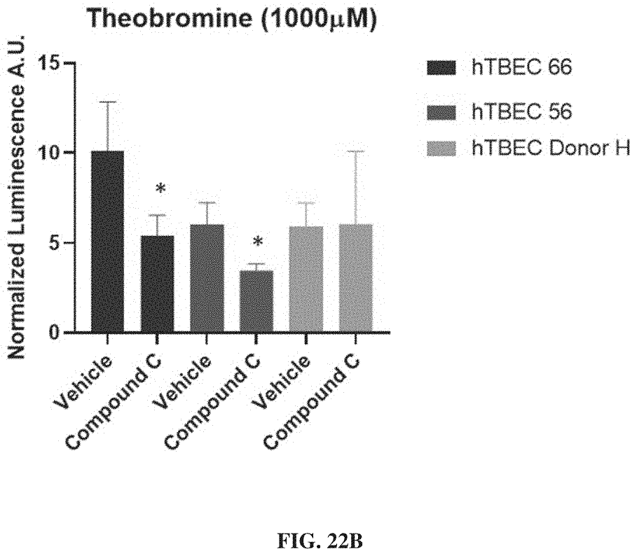

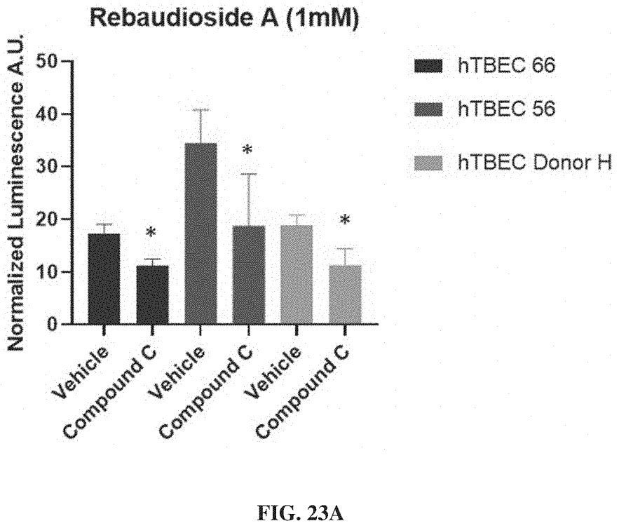

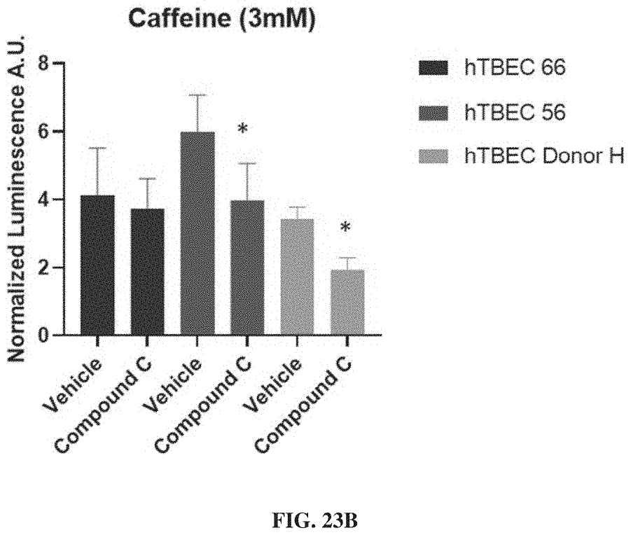

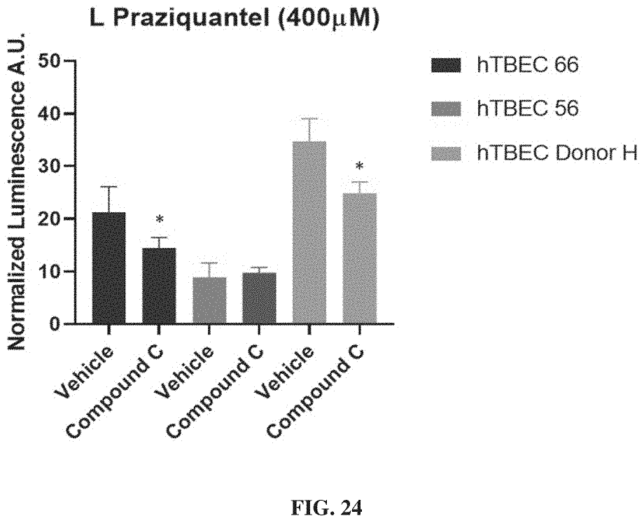

BB13 was further evaluated in all three hTBEC cultures. BB13 consistently showed inhibitory activity against 100 μM Dextromethorphan-HBr (FIG. 22A), as well as against 1,0001 μM Theobromine (FIG. 22B), 1 mM Rebaudioside A (FIG. 23A), 3 mM Caffeine (FIG. 23B), and 400 μM L-Praziquantel (FIG. 24).

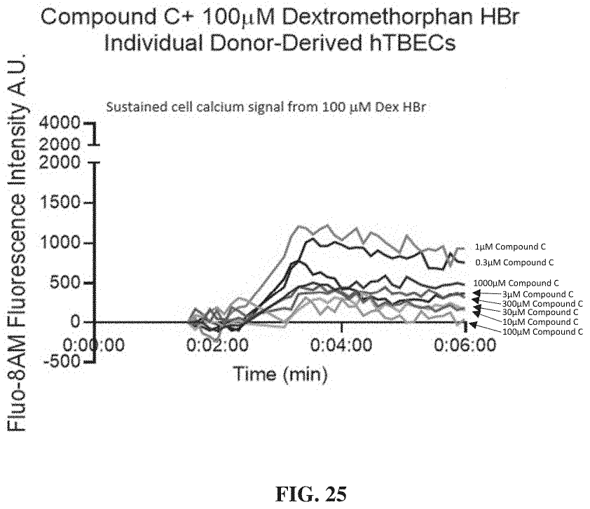

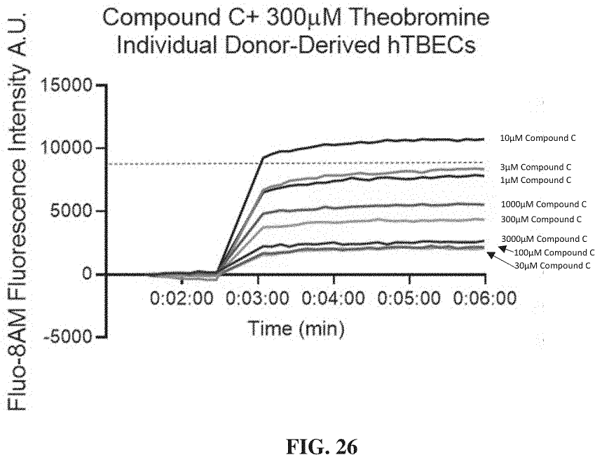

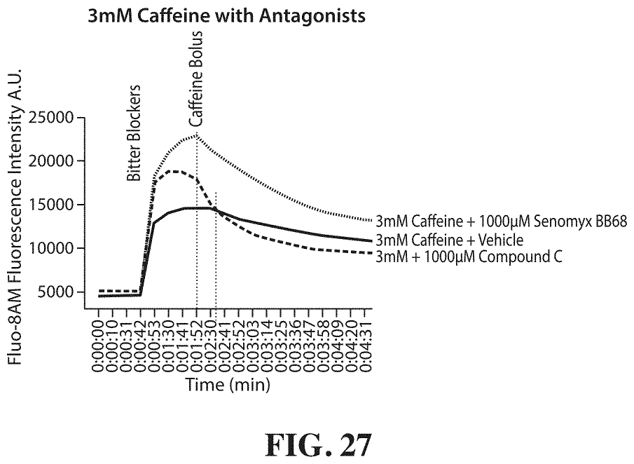

Calcium mobilization response following treatment of 100 μM Dextromethorphan-HBr, 3000 μM Theobromine, and 3 mM Caffeine was evaluated in individual donor-derived hTBECs. At high concentrations, BB13 inhibited calcium mobilization induced by Dextromethorphan-HBr (FIG. 25), as well as that induced by Theobromine (FIG. 26), and Caffeine (FIG. 27).

Sequences of Interest
TcCGT1 Protein
SEQ ID NO: 1
MEKSNPNSTSKPHVFLLASPGMGHLIPFLELSKRLVTLNTLQVTLFIVSN
EATKARSHLMESSNNFHPDLELVDLTPANLSELLSTDATVFKRIFLITQA
AIKDLESRISSMSTPPAALIVDVFSMDAFPVADRFGIKKYVFVTLNAWFL
ALTTYVRTLDREIEGEYVDLPEPIAIPGCKPLRPEDVFDPMLSRSSDGYR
PYLGMSERLTKADGLLLNTWEALEPVSLKALRENEKLNQIMTPPLYPVGP
VARTTVQEVVGNECLDWLSKQPTESVLYVALGSGGIISYKQMTELAWGLE
MSRQRFIWVVRLPTMEKDGACRFFSDVNVKGPLEYLPEGFLDRNKELGMV
LPNWGPQDAILAHPSTGGFLSHCGWNSSLESIVNGVPVIAWPLYAEQKMN
ATLLTEELGVAVRPEVLPTKAVVSRDEIEKMVRRVIESKEGKMKRNRARS
VOSDALKAIEKGGSSYNTLIEVAKEFEKNHKVL
TCCGT1 DNA
SEQ ID NO: 2
ATGGAGAAGTCAAATCCAAATTCGACTICAAAGCCGCATGTATTCCTGCT
GGCGAGCCCGGGGATGGGCCACTTAATCCCGTTTCTCGAGTTATCAAAGC
GGCTGGTGACCTTAAATACCTTACAGGTAACCTTATTCATCGTATCAAAC
GAAGCTACTAAAGCGCGGTCACATCTGATGGAATCATCAAATAATTTCCA
CCCAGATCTGGAATTAGTGGATTTAACCCCGGCGAATTTATCAGAGTTAC
TGAGCACTGACGCGACCGTATTCAAACGGATCTTCTTAATCACCCAGGCT
GCTATTAAAGACCTGGAATCACGCATTAGCTCAATGAGTACCCCGCCGGC
GGCGTTAATCGTAGACGTATTCTCGATGGACGCCTTTCCGGTGGCGGATC
GTTTTGGCATCAAGAAGTATGTCTTTGTGACCTTAAACGCGTGGTTTCTG
GCGCTGACCACCTACGTACGGACCCTGGATCGGGAAATTGAAGGCGAGTA
TGTGGATCTGCCGGAGCCGATTGCGATCCCGGGCTGCAAACCGTTACGGC
CAGAGGACGTGTTTGACCCGATGCTGAGCCGTAGCAGCGATGGGTATCGC
CCGTACCTGGGGATGAGCGAGCGTTTAACCAAGGCGGATGGGCTGCTGCT
GAATACCTGGGAAGCCTTAGAGCCAGTCTCGCTGAAGGCGCTGCGCGAAA
ACGAGAAATTAAACCAAATCATGACTCCGCCGCTGTACCCAGTGGGCCCG
GTCGCGCGGACCACCGTCCAAGAGGTCGTCGGGAACGAGTGTCTGGATTG
GTTATCGAAGCAGCCAACCGAGICAGTACTGTACGTAGCCCTGGGCAGCG
GCGGGATCATTTCATACAAACAGATGACTGAGTTAGCGTGGGGCCTGGAA
ATGTCGCGGCAGCGGTTTATCTGGGTCGTGCGGTTACCAACTATGGAGAA
AGACGGGGCCTGCCGGTTCTTTTCAGACGTGAACGTCAAAGGGCCGCTGG
AATACCTGCCAGAAGGGTTCCTGGACCGGAACAAGGAGCTGGGCATGGTC
TTACCGAACTGGGGGCCGCAGGACGCCATCCTGGCTCATCCGAGTACTGG
CGGCTTTCTCTCACATTGCGGCTGGAACTCATCACTGGAGTCGATTGTCA
ATGGCGTCCCGGTCATCGCGTGGCCGCTGTACGCGGAGCAGAAAATGAAT
GCTACCCTGCTGACCGAAGAGTTAGGCGTGGCCGTACGGCCGGAAGTCTT
ACCGACTAAGGCGGTCGICAGCCGTGATGAGATCGAGAAAATGGTCCGTC
GCGTAATCGAAAGCAAGGAAGGGAAAATGAAGCGCAACCGCGCTCGCAGC
GTACAAAGCGATGCGCTGAAAGCGATTGAAAAGGGGGGGTCAAGCTATAA
CACCTTAATCGAGGTCGCAAAGGAGTTCGAGAAGAACCACAAAGTACTG
UGT73B2 Protein
SEQ ID NO: 3
MGSDHHHRKLHVMFFPFMAYGHMIPTLDMAKLFSSRGAKSTILTTSLNSK
ILQKPIDTFKNLNPGLEIDIQIFNFPCVELGLPEGCENVDFFTSNNNDDK
NEMIVKFFFSTRFFKDQLEKLLGTTRPDCLIADMFFPWATEAAGKFNVPR
LVFHGTGYFSLCAGYCIGVHKPQKRVASSSEPFVIPELPGNIVITEEQII
DGDGESDMGKFMTEVRESEVKSSGVVLNSFYELEHDYADFYKSCVQKRAW
HIGPLSVYNRGFEEKAERGKKANIDEAECLKWLDSKKPNSVIYVSFGSVA
FFKNEQLFEIAAGLEASGTSFIWVVRKTKDDREEWLPEGFEERVKGKGMI
IRGWAPQVLILDHQATGGFVTHCGWNSLLEGVAAGLPMVTWPVGAEQFYN
EKLVTQVLRTGVSVGASKHMKVMMGDFISREKVDKAVREVLAGEAAEERR
RRAKKLAAMAKAAVEEGGSSFNDLNSFMEEFSS
UGT73B2 DNA
SEQ ID NO: 4
ATGGGTTCAGACCACCACCACCGCAAACTGCACGTTATGTTCTTCCCGTT
TATGGCTTACGGCCACATGATTCCGACGCTGGATATGGCGAAACTGTTCA
GCTCTCGTGGTGCCAAAAGCACCATCCTGACCACGTCTCTGAATAGTAAA
ATCCTGCAGAAACCGATTGATACGTTTAAAAATCTGAACCCGGGCCTGGA
AATTGACATCCAAATTTTCAACTTTCCGTGCGTTGAACTGGGCCTGCCGG
AAGGTTGTGAAAATGTCGATTTCTTTACCTCCAACAATAACGATGACAAA
AACGAAATGATCGTGAAATTTTTCTTTTCAACGCGTTTCTTTAAAGATCA
GCTGGAAAAACTGCTGGGTACCACGCGCCCGGATTGCCTGATTGCGGACA
TGTTCTTTCCGTGGGCCACCGAAGCGGCCGGCAAATTTAATGTGCCGCGT
CTGGTTTTCCATGGCACGGGTTATTTTTCGCTGTGCGCAGGCTACTGTAT
CGGTGTGCACAAACCGCAGAAACGCGTTGCTAGTTCCTCAGAACCGTTCG
TCATTCCGGAACTGCCGGGTAACATCGTGATCACCGAAGAACAAATCATC
GATGGCGACGGTGAATCAGATATGGGTAAATTTATGACCGAAGTTCGTGA
ATCGGAAGTCAAATCGAGCGGCGTGGTTCTGAACAGCTTCTATGAACTGG
AACATGATTATGCGGACTTTTACAAATCTTGCGTCCAGAAACGCGCCTGG
CACATTGGCCCGCTGAGTGTTTACAATCGTGGTTTTGAAGAAAAAGCGGA
ACGCGGCAAAAAAGCGAACATCGATGAAGCCGAATGTCTGAAATGGCTGG
ACTCCAAAAAACCGAACAGCGTGATTTATGTTTCCTTCGGCTCAGTTGCC
TTCTTTAAAAACGAACAGCTGTTTGAAATCGCAGCTGGCCTGGAAGCATC
GGGTACCAGCTTCATTTGGGTCGTGCGTAAAACGAAAGATGACCGCGAAG
AATGGCTGCCGGAAGGTTTTGAAGAACGTGTGAAAGGCAAGGGTATGATT
ATCCGTGGTTGGGCACCGCAGGTGCTGATCCTGGATCATCAAGCTACCGG
CGGTTTCGTTACGCACTGTGGTTGGAACAGCCTGCTGGAAGGCGTGGCAG
CAGGTCTGCCGATGGTCACCTGGCCGGTGGGCGCGGAACAGTTTTACAAC
GAAAAACTGGTCACCCAAGTGCTGCGCACGGGCGTTTCTGTCGGTGCCAG
TAAACACATGAAAGTGATGATGGGTGATTTCATTAGTCGTGAAAAAGTTG
ACAAAGCAGTTCGCGAAGTCCTGGCTGGCGAAGCAGCTGAAGAACGTCGC
CGTCGCGCGAAAAAACTGGCGGCCATGGCTAAAGCAGCTGTGGAAGAAGG
CGGCAGCAGTTTTAATGACCTGAATAGTTTTATGGAAGAATTTAGTTCGT
GA
BcGT1 Protein
SEQ ID NO: 5
MANVLVINFPGEGHINPTLAIVSELIRRGETVVSYCIEDYRKKIEATGAQ
FRVFENFLSQINIMERVNEGGSPLTMLSHMMEASERIVTQIVEETKGEKY
DYLIYDNHFPVGRIIANVLKLPSVSSCTTFAFNQYITFNDEHESREVDET
NPLYQSCLAGMEKWNKQYGMKCNSMYDIMNHPGDITIVYTSKEYQPRSDV
FDESYKFVGPSIATRKEVGSFPMEDLKDEKLIFISMGTVFNEQPELYEKC
FEAFKDVEATVVLVVGKKINISQFENIPNNFKLYNYVPQLELLQYADVFV
THGGMNSSSEALYYGVPLVVIPVTGDQPLVAKRVNEVGAGIRLNRKELTS
EMLRESVKKVMDDVTFKEKSRKVGESLRNAGGYNRAVDEILKMNSYSKLK
BcGT1 DNA
SEQ ID NO: 6
ATGGCAAACGTACTCGTAATAAATTTCCCTGGAGAAGGTCATATAAATCC
GACTTTAGCTATTGTAAGTGAGTTAATTCGGCGAGGGGAGACAGTTGTTT
CGTATTGTATTGAAGATTATAGAAAGAAGATTGAAGCAACAGGTGCACAA
TTCCGAGTGTTTGAGAATTTCCTCTCTCAAATTAATATTATGGAGCGAGT
AAATGAAGGTGGGAGTCCTTTGACGATGCTGTCTCACATGATGGAAGCAT
CAGAACGTATTGTTACTCAAATTGTAGAAGAAACAAAAGGGGAAAAGTAC
GATTATTTGATATATGATAATCACTTTCCAGTAGGACGTATTATAGCCAA
TGTTTTAAAGTTACCTAGTGTTTCTTCTTGTACAACGTTTGCTTTTAATC
AGTACATTACTTTTAACGATGAACATGAATCAAGAGAAGTAGATGAAACG
AATCCATTGTATCAATCTTGTTTAGCGGGAATGGAAAAATGGAACAAACA
GTATGGAATGAAATGTAATAGTATGTATGATATTATGAACCATCCTGGTG
ATATTACAATTGTGTATACTTCAAAGGAATATCAGCCGCGTTCAGATGTA
TTCGATGAATCGTATAAGTTTGTTGGCCCATCAATTGCTACTCGAAAAGA
AGTAGGTAGCTTTCCTATGGAAGATTTAAAAGATGAAAAATTGATTTTCA
TTTCTATGGGAACAGTTTTTAATGAACAACCTGAGTTATATGAAAAATGT
TTTGAAGCGTTTAAAGATGTAGAAGCGACAGTCGTATTAGTTGTTGGTAA
GAAGATAAATTAGAACTATTACAGTATGCTGATGTATTCGTAACACACGG
CGGTATGAATAGTTCAAGTGAAGCACTATATTACGGTGTCCCGTTAGTTG
TAATTCCGGTAACAGGAGATCAGCCTTTAGTTGCGAAACGAGTAAATGAA
GTAGGGGCTGGAATAAGGCTTAATCGCAAAGAATTAACTTCTGAAATGTT
ACGTGAGTCTGTAAAGAAAGTGATGGATGATGTAACGTTTAAGGAAAAAA
GTCGTAAAGTTGGAGAGTCACTTCGAAATGCTGGTGGTTATAATAGGGCA
GTTGATGAAATATTAAAAATGAATTCATACTCAAAACTTAAATAA

1. A method of reducing or blocking the bitter taste of an orally consumable composition comprising one or more bitter tastants, the method comprising adding to the orally consumable composition an effective amount of a bitter blocker selected from the group consisting of eriodictyol-8-C-β-glucoside, homoeriodictyol 4′-O-glucoside, and homoeriodictyol 7-O-glucoside, optionally, such that the bitter taste of the orally consumable composition is reduced by at least 50%.

2. The method of claim 1, wherein the one or more bitter tastants are selected from the group consisting of caffeine, bitter methylxanthines, theobromine, rebaudioside A, a B vitamin, cannabidiol, tetrahydrocannabinol, nicotine, dextromethorphan, dextromethorphan hydrobromide, chlorhexidine, guaifenesin, pseudoephedrine, atorvastatin, aspirin, acetaminophen, diphenhydramine, doxylamine, sildenafil citrate, and loperamide.

3. The method of claim 1, wherein the orally consumable composition comprises at least 100 mg/L of the one or more bitter tastants.

4. The method of claim 1, wherein the bitter taste of the orally consumable composition is reduced by at least 50%, 60%, or 80%.

5.-8. (canceled)

9. An orally consumable composition comprising: a) one or more bitter tastants; and b) a bitter blocker selected from the group consisting of eriodictyol-8-C-β-glucoside, homoeriodictyol 4′-O-glucoside, and homoeriodictyol 7-O-glucoside; optionally, wherein the bitter blocker is present in the orally consumable composition in a concentration between about 10 ppm and about 200 ppm.

10. The composition of claim 9, wherein the one or more bitter tastants are selected from the group consisting of: caffeine, bitter methylxanthines, theobromine, rebaudioside A, a B vitamin, cannabidiol, tetrahydrocannabinol, nicotine, dextromethorphan, dextromethorphan hydrobromide, chlorhexidine, guaifenesin, pseudoephedrine, atorvastatin, aspirin, acetaminophen, diphenhydramine, doxylamine, sildenafil citrate, and loperamide.

11. The composition of claim or claim 9, comprising at least 100 mg/L of the one or more bitter tastants.

12. The composition of claim 9, wherein the composition is selected from the group consisting of a food product, a functional food, a pharmaceutical, a dietary supplement, a dental hygiene composition, a food grade gel composition, a cosmetic product, and a flavoring product.

13. The composition of claim 9, wherein the composition is a beverage product selected from the group consisting of coffee, tea, fermented tea, a dairy beverage, a plant-based milk beverage, an alcoholic beverage, flavored water, vitamin water, fruit juice, and an energy drink.

14.-16. (canceled)

17. The method or use of claim 1, wherein the one or more bitter tastants are in a orally consumable composition.

18. (canceled)

19. A method of preparing a flavonoid glycoside, the method comprising incubating a reaction mixture comprising:
(i) a) uridine diphosphate-glucose, b) eriodictyol as a substrate, and c) a glycosyltransferase comprising an amino acid sequence having at least 80% sequence identity to SEQ ID NO: 1, wherein a glucose is covalently coupled to the eriodictyol substrate to produce eriodictyol-8-C-β-glucoside, optionally wherein the glycosyltransferase comprises the amino acid sequence of SEQ ID NO: 1, or
(ii) a) uridine diphosphate-glucose, b) homoeriodictyol as a substrate, and c) a glycosyltransferase comprising an amino acid sequence having at least 80% sequence identity to SEQ ID NO: 3 or SEQ ID NO: 5, wherein a glucose is covalently coupled to the homoeriodictyol substrate to produce homoeriodictyol 4′-O-glucoside and/or homoeriodictyol 7-O-glucoside, optionally wherein the glycosyltransferase comprises the amino acid sequence of SEQ ID NO: 3 or SEQ ID NO: 5.

20. (canceled)

21. The method of claim 19, wherein the reaction mixture is in vitro.

22. The method of claim 19, wherein the reaction mixture is a cell-based reaction mixture.

23. The method of claim 22, wherein the cell-based reaction mixture comprises a cell comprising a polynucleotide encoding the glycosyltransferase, wherein the polynucleotide comprises a nucleotide sequence that is at least 90% identical to any one of SEQ ID NOs: 2, 4, 6.

24.-29. (canceled)

30. A reaction mixture comprising:
(a) uridine diphosphate-glucose,
(b) a natural flavanone, and
(c) a host cell comprising a glycosyltransferase comprising an amino acid sequence having at least 80% sequence identity to any one of SEQ ID NOs: 1, 3, 5.

31. The reaction mixture of claim 30, wherein the natural flavanone is homoeriodictyol, eriodictyol, or combinations thereof.

32. The reaction mixture of claim 30, wherein the host cell is a bacterial cell.

33. The reaction mixture of claim 30, wherein the host cell is an Escherichia coli (E. coli) cell.

34. (canceled)

35. The reaction mixture of claim 30, further comprising: eriodictyol-8-C-β-glucoside, homoeriodictyol 4′-O-glucoside, homoeriodictyol 7-O-glucoside, or combinations thereof.

36. A compound produced by the method of claim 19.

37.-38. (canceled)


SEQUENCE LISTING (6 sequences)

SEQ ID NO: 1 - protein, 483 aa, unidentified
  REGION 1..483: note = Trollius chinensis
MEKSNPNSTSKPHVFLLASPGMGHLIPFLELSKRLVTLNTLQVTLFIVSNEATKARSHLMESSNNFHPDLELVDLTPANLSELLSTDATVFKRIFLITQAAIKDLESRISSMSTPPAALIVDVFSMDAFPVADRFGIKKYVFVTLNAWFLALTTYVRTLDREIEGEYVDLPEPIAIPGCKPLRPEDVFDPMLSRSSDGYRPYLGMSERLTKADGLLLNTWEALEPVSLKALRENEKLNQIMTPPLYPVGPVARTTVQEVVGNECLDWLSKQPTESVLYVALGSGGIISYKQMTELAWGLEMSRQRFIWVVRLPTMEKDGACRFFSDVNVKGPLEYLPEGFLDRNKELGMVLPNWGPQDAILAHPSTGGFLSHCGWNSSLESIVNGVPVIAWPLYAEQKMNATLLTEELGVAVRPEVLPTKAVVSRDEIEKMVRRVIESKEGKMKRNRARSVQSDALKAIEKGGSSYNTLIEVAKEFEKNHKVL

SEQ ID NO: 2 - DNA, 1449 bp, unidentified
  misc_feature 1..1449: note = Trollius chinensis
  source 1..1449: mol_type = genomic DNA
atggagaagtcaaatccaaattcgacttcaaagccgcatgtattcctgctggcgagcccggggatgggccacttaatcccgtttctcgagttatcaaagcggctggtgaccttaaataccttacaggtaaccttattcatcgtatcaaacgaagctactaaagcgcggtcacatctgatggaatcatcaaataatttccacccagatctggaattagtggatttaaccccggcgaatttatcagagttactgagcactgacgcgaccgtattcaaacggatcttcttaatcacccaggctgctattaaagacctggaatcacgcattagctcaatgagtaccccgccggcggcgttaatcgtagacgtattctcgatggacgcctttccggtggcggatcgttttggcatcaagaagtatgtctttgtgaccttaaacgcgtggtttctggcgctgaccacctacgtacggaccctggatcgggaaattgaaggcgagtatgtggatctgccggagccgattgcgatcccgggctgcaaaccgttacggccagaggacgtgtttgacccgatgctgagccgtagcagcgatgggtatcgcccgtacctggggatgagcgagcgtttaaccaaggcggatgggctgctgctgaatacctgggaagccttagagccagtctcgctgaaggcgctgcgcgaaaacgagaaattaaaccaaatcatgactccgccgctgtacccagtgggcccggtcgcgcggaccaccgtccaagaggtcgtcgggaacgagtgtctggattggttatcgaagcagccaaccgagtcagtactgtacgtagccctgggcagcggcgggatcatttcatacaaacagatgactgagttagcgtggggcctggaaatgtcgcggcagcggtttatctgggtcgtgcggttaccaactatggagaaagacggggcctgccggttcttttcagacgtgaacgtcaaagggccgctggaatacctgccagaagggttcctggaccggaacaaggagctgggcatggtcttaccgaactgggggccgcaggacgccatcctggctcatccgagtactggcggctttctctcacattgcggctggaactcatcactggagtcgattgtcaatggcgtcccggtcatcgcgtggccgctgtacgcggagcagaaaatgaatgctaccctgctgaccgaagagttaggcgtggccgtacggccggaagtcttaccgactaaggcggtcgtcagccgtgatgagatcgagaaaatggtccgtcgcgtaatcgaaagcaaggaagggaaaatgaagcgcaaccgcgctcgcagcgtacaaagcgatgcgctgaaagcgattgaaaagggcgggtcaagctataacaccttaatcgaggtcgcaaaggagttcgagaagaaccacaaagtactg

SEQ ID NO: 3 - protein, 483 aa, Arabidopsis thaliana
MGSDHHHRKLHVMFFPFMAYGHMIPTLDMAKLFSSRGAKSTILTTSLNSKILQKPIDTFKNLNPGLEIDIQIFNFPCVELGLPEGCENVDFFTSNNNDDKNEMIVKFFFSTRFFKDQLEKLLGTTRPDCLIADMFFPWATEAAGKFNVPRLVFHGTGYFSLCAGYCIGVHKPQKRVASSSEPFVIPELPGNIVITEEQIIDGDGESDMGKFMTEVRESEVKSSGVVLNSFYELEHDYADFYKSCVQKRAWHIGPLSVYNRGFEEKAERGKKANIDEAECLKWLDSKKPNSVIYVSFGSVAFFKNEQLFEIAAGLEASGTSFIWVVRKTKDDREEWLPEGFEERVKGKGMIIRGWAPQVLILDHQATGGFVTHCGWNSLLEGVAAGLPMVTWPVGAEQFYNEKLVTQVLRTGVSVGASKHMKVMMGDFISREKVDKAVREVLAGEAAEERRRRAKKLAAMAKAAVEEGGSSFNDLNSFMEEFSS

SEQ ID NO: 4 - DNA, 1452 bp, Arabidopsis thaliana
  source 1..1452: mol_type = genomic DNA
atgggttcagaccaccaccaccgcaaactgcacgttatgttcttcccgtttatggcttacggccacatgattccgacgctggatatggcgaaactgttcagctctcgtggtgccaaaagcaccatcctgaccacgtctctgaatagtaaaatcctgcagaaaccgattgatacgtttaaaaatctgaacccgggcctggaaattgacatccaaattttcaactttccgtgcgttgaactgggcctgccggaaggttgtgaaaatgtcgatttctttacctccaacaataacgatgacaaaaacgaaatgatcgtgaaatttttcttttcaacgcgtttctttaaagatcagctggaaaaactgctgggtaccacgcgcccggattgcctgattgcggacatgttctttccgtgggccaccgaagcggccggcaaatttaatgtgccgcgtctggttttccatggcacgggttatttttcgctgtgcgcaggctactgtatcggtgtgcacaaaccgcagaaacgcgttgctagttcctcagaaccgttcgtcattccggaactgccgggtaacatcgtgatcaccgaagaacaaatcatcgatggcgacggtgaatcagatatgggtaaatttatgaccgaagttcgtgaatcggaagtcaaatcgagcggcgtggttctgaacagcttctatgaactggaacatgattatgcggacttttacaaatcttgcgtccagaaacgcgcctggcacattggcccgctgagtgtttacaatcgtggttttgaagaaaaagcggaacgcggcaaaaaagcgaacatcgatgaagccgaatgtctgaaatggctggactccaaaaaaccgaacagcgtgatttatgtttccttcggctcagttgccttctttaaaaacgaacagctgtttgaaatcgcagctggcctggaagcatcgggtaccagcttcatttgggtcgtgcgtaaaacgaaagatgaccgcgaagaatggctgccggaaggttttgaagaacgtgtgaaaggcaagggtatgattatccgtggttgggcaccgcaggtgctgatcctggatcatcaagctaccggcggtttcgttacgcactgtggttggaacagcctgctggaaggcgtggcagcaggtctgccgatggtcacctggccggtgggcgcggaacagttttacaacgaaaaactggtcacccaagtgctgcgcacgggcgtttctgtcggtgccagtaaacacatgaaagtgatgatgggtgatttcattagtcgtgaaaaagttgacaaagcagttcgcgaagtcctggctggcgaagcagctgaagaacgtcgccgtcgcgcgaaaaaactggcggccatggctaaagcagctgtggaagaaggcggcagcagttttaatgacctgaatagttttatggaagaatttagttcgtga

SEQ ID NO: 5 - protein, 400 aa, Bacillus cereus
MANVLVINFPGEGHINPTLAIVSELIRRGETVVSYCIEDYRKKIEATGAQFRVFENFLSQINIMERVNEGGSPLTMLSHMMEASERIVTQIVEETKGEKYDYLIYDNHFPVGRIIANVLKLPSVSSCTTFAFNQYITFNDEHESREVDETNPLYQSCLAGMEKWNKQYGMKCNSMYDIMNHPGDITIVYTSKEYQPRSDVFDESYKFVGPSIATRKEVGSFPMEDLKDEKLIFISMGTVFNEQPELYEKCFEAFKDVEATVVLVVGKKINISQFENIPNNFKLYNYVPQLELLQYADVFVTHGGMNSSSEALYYGVPLVVIPVTGDQPLVAKRVNEVGAGIRLNRKELTSEMLRESVKKVMDDVTFKEKSRKVGESLRNAGGYNRAVDEILKMNSYSKLK

SEQ ID NO: 6 - DNA, 1203 bp, Bacillus cereus
  source 1..1203: mol_type = genomic DNA
atggcaaacgtactcgtaataaatttccctggagaaggtcatataaatccgactttagctattgtaagtgagttaattcggcgaggggagacagttgtttcgtattgtattgaagattatagaaagaagattgaagcaacaggtgcacaattccgagtgtttgagaatttcctctctcaaattaatattatggagcgagtaaatgaaggtgggagtcctttgacgatgctgtctcacatgatggaagcatcagaacgtattgttactcaaattgtagaagaaacaaaaggggaaaagtacgattatttgatatatgataatcactttccagtaggacgtattatagccaatgttttaaagttacctagtgtttcttcttgtacaacgtttgcttttaatcagtacattacttttaacgatgaacatgaatcaagagaagtagatgaaacgaatccattgtatcaatcttgtttagcgggaatggaaaaatggaacaaacagtatggaatgaaatgtaatagtatgtatgatattatgaaccatcctggtgatattacaattgtgtatacttcaaaggaatatcagccgcgttcagatgtattcgatgaatcgtataagtttgttggcccatcaattgctactcgaaaagaagtaggtagctttcctatggaagatttaaaagatgaaaaattgattttcatttctatgggaacagtttttaatgaacaacctgagttatatgaaaaatgttttgaagcgtttaaagatgtagaagcgacagtcgtattagttgttggtaagaagataaatataagtcaatttgaaaacattccgaataactttaagttgtataattatgtgccgcaattagaactattacagtatgctgatgtattcgtaacacacggcggtatgaatagttcaagtgaagcactatattacggtgtcccgttagttgtaattccggtaacaggagatcagcctttagttgcgaaacgagtaaatgaagtaggggctggaataaggcttaatcgcaaagaattaacttctgaaatgttacgtgagtctgtaaagaaagtgatggatgatgtaacgtttaaggaaaaaagtcgtaaagttggagagtcacttcgaaatgctggtggttataatagggcagttgatgaaatattaaaaatgaattcatactcaaaacttaaataa